(19) United States
(12) Patent Application Publication
(10) Pub. No.: US 2024/0148523 A1
(43) Pub. Date: May 9, 2024

(54) System and Method for an Improved Redundant Crossfire Circuit in a Fully Integrated Neurostimulation Device and Its Use in Neurotherapy

(71) Applicant: Regents of the University of Minnesota, Minneapolis, MN (US)

(72) Inventors: Zhi Yang, Minneapolis, MN (US); Anh Tuan Nguyen, Minneapolis, MN (US); Diu Khue Luu, Minneapolis, MN (US)

(21) Appl. No.: 18/396,610

(22) Filed: Dec. 26, 2023

Related U.S. Application Data
(63) Continuation-in-part of application No. PCT/US2022/035016, filed on Jun. 24, 2022.
(60) Provisional application No. 63/214,623, filed on Jun. 24, 2021.

(51) Int. Cl. A61F 2/72; A61F 2/58; G06F 3/01

(52) U.S. Cl. CPC A61F 2/72; A61F 2/583; G06F 3/015; A61F 2002/6827

(57) ABSTRACT

A neurostimulator incorporating a novel chip design that uses the principle of redundant signal crossfiring to overcome electronic component mismatch error in general and transistor mismatch error in particular, to yield superior quality neurostimulation signal generation, useful in enhancing the bidirectional human-machine interface in prosthesis operation for the restoration of somatosensation for an amputee; and an improvement thereof additionally comprising a digital-to-analog converter device, that includes a number of unit cells, each unit cell being associated with a unit cell size indicating manufacturing specifications of the unit cell, and that further includes a plurality of switches, each being coupled to a component, and an output electrode coupled to the plurality of switches, and wherein the digital-to-analog converter device is configured to output an output signal at the output electrode.

### CROSS-REFERENCE TO RELATED APPLICATIONS

The present application is a continuation-in-part application that is based on, claims priority to, and incorporates by reference in its entirety for all purposes, PCT Application PCT/US22/35016, filed Jun. 24, 2022, which claims priority to U.S. Provisional Application Ser. No. 63/214,623, filed Jun. 24, 2021, and each is entitled: “SYSTEM AND METHOD FOR AN IMPROVED REDUNDANT CROSSFIRE CIRCUIT IN A FULLY INTEGRATED NEUROSTIMULATION DEVICE AND ITS USE IN NEUROTHERAPY.”

### STATEMENT ON FEDERALLY SPONSORED RESEARCH

This invention was made with government support under Agency Grant Number 1845709 awarded by the National Science Foundation, and Agency Grant Number 1R21NS111214-01 awarded by the National Institute of Health. The government has certain rights in the invention.

### FIELD OF THE INVENTION

This invention generally concerns systems and methods for neuromodulation, and, more specifically, concerns miniaturized neurostimulators in integrated circuits that convert and exploit the problem of mismatch error into an effective means of achieving super-resolution signal accuracy in neuromodulation.

This invention also generally concerns the field of electrical converters including digital-to-analog converters (DACs) and analog-to-digital converters (ADCs). More particularly, the invention relates to super-resolution DACs using redundant sensing techniques.

### BACKGROUND OF THE INVENTION

Electrical stimulation has been used in research and in therapeutics for probing neural circuitry and identifying networks of neurons for many years. Despite its extensive use, the mechanism of electrical stimulation on the nervous system remains poorly understood, and the results of applying novel approaches to such therapeutic stimulation cannot be predicted with a reasonable degree of certainty by those of ordinary skill in the art due to the uncertainty of the outcome prior to pre-clinical experimental research on animal models and clinical experimental development on human subjects. There exists a need for an integrated hardware and software platform system that can sense, analyze, and modulate neural signals, and to define, incorporate, and transmit neural feedback-influenced electrical signals in order to improve electrical stimulation outcomes. Currently available or known electrical stimulation devices do not adequately sense nerve neural signals, being unable to satisfactorily overcome the fact that nerve neural signals are smaller than signals measured in the brain, meaning that existing recording technology is inadequate for resolving nerve neural signals from signals measured in the brain, which constitute sources of signal noise.

Signal Noise. Noise, in the context here of signal processing, is defined as a general term for unwanted (and, in general, unknown, unknowable, or unpredictable) modifications that a signal may suffer during capture, storage, transmission, processing, and conversion of the signal for purposes of recording and analyzing the signal of interest. This problem is made even worse when a prior art neurostimulation device is a non-dermal penetrating type of cuff electrode. When penetrating or intraneural electrodes are used to address this problem, nerve signal quality is improved at early time points, but the signal decays over time due to foreign body host response processes (e.g. inflammatory responses) and due to the micro-motion of the position of the tethered electrodes, in relation to the surrounding soft tissues into which the electrodes are embedded. Additionally, there is the inherent problem that whenever electrical stimulation is applied, there are large, sustained noise artifacts. Prior art attempts to address this have been through efforts at blocking or blanking out the noise, rather than subtracting out the noisy signals, thereby losing neural responses that may be measured locally with greater accuracy. An integrated approach as utilized in the present invention for developing a superior neuromodulation device therefore will comprise the four strategies of: (1) developing ultra-low noise hardware technology for sensing neural signals, most preferably (but not exclusively) without requiring electrode penetration into a nerve; (2) developing a deep neural network-based algorithm to analyze and decode signals; (3) developing hardware technology for both selective amplification of signals of interest and for removing stimulation signal residual charges and noise artifacts to support simultaneous recording and stimulation of a nerve; and (4) developing prediction models of therapeutic outcomes, based on stimulation parameters and neural signals. The present invention will, in its most preferred embodiment, play a major part in signal analysis and decoding, operating within the overall architecture of this four-part integrated approach.

Poor signal-to-noise ratio (SNR) of neural signals in nerves. Epineural electrodes, such as cuffs, provide a robust and stable interface for recording whole-nerve neural activity, but the electrical isolation of the epineurium/perineurium reduces the magnitude of signals, resulting in a low SNR. In particular, the biological noise in epineural electrodes can significantly distort recordings and even bury the neural signals, if the precision of the recorder is not sufficiently high. Post-signal processing to remove biological noise often creates new noise artifacts that can be confounded with nerve signals. Due to different chemical compounds, cell tissue environments, and tissue responses to implants, the electrode-tissue interface noise can be very different from a measurement in a bench testing apparatus. Several models and more recent measurement data suggest that a large portion of electrode noise is non-Ohmic (Johnson-Nyquist noise, or thermal noise), which is the electronic noise generated by the thermal agitation of the charge carriers (usually the electrons) inside an electrical conductor at equilibrium, which happens regardless of any applied voltage. For such non-Ohmic noise, the sensing electronics can play a role in reducing the electrode noise. The amount of electronics noise in nerve recordings can be quite large due to elevated filter noise. For example, when recording a nerve in the abdomen, the constant motion of viscera causes large amplitude-motion artifacts. To avoid saturating electronics, an add-on analog filter with a corner frequency at least ten times higher than the artifact frequency is required. This ten-fold requirement increases the analog filter noise in the neural signal band up to tens of μV and beyond. Therefore, it can be seen that the battle to improve neural recording signal quality goes beyond the optimization of individual noise components.

Inabilities to extract signals of individual fascicles and inabilities to develop chronic stability. Noninvasive nerve interface approaches provide small signals from a highly limited number of electrodes. Current technologies have demonstrated that recordings of compound nerve action potentials (CNAPs), mostly from exposed nerves, are sufficiently greater than the background noise activities, but CNAPs constitute such a volley of synchronous action potentials that it is not possible to deconvolute them into individual fiber activities, which is required to perform neural decoding. Spontaneous or less-synchronized neural activities from a nerve are much smaller than CNAPs, and where it is particularly true of cuff electrodes, it is seen that such signals are further attenuated by the physical presence of the nerve sheath. For example, it has been confirmed in the prior art that after using neuromuscular blockers, surface canine vagus nerve signals are in the range of microvolts to tens of microvolts for de-sheathed nerves, but are even smaller (estimated at less than 10 μV) when the epineurium was intact during surface recordings. A nerve is an enclosed, cable-like bundle of many myelinated nerve fibers, structured as sheaths: the endoneurium is a layer of connective tissue around the myelin sheath of each fiber. The endoneuria with their enclosed nerve fibers are bundled into nerve fascicles, and each fascicle is within its own protective sheath called a perineurium. Multiple fascicles are bundled within yet another sheath, the epineurium. These multiple protective sheaths isolate fibers, making it extremely hard to record and then modulate signals on individual fibers from the outside of the nerve. Invasive intraneural electrodes may produce detectable signals at early time points, but signal decay over time due to tissue response has remained a problem. Initial insertion of the electrodes causes an early host immune system foreign body response leading to tissue encapsulation, with extensive tissue damage resulting from the micro-motion of the tethered electrodes relative to the soft tissue. Due to a lack of chronic stability, intraneural electrodes pose a significant long-term usage challenge. Therefore, there is still a need to pursue non-penetrating neural interfaces with significantly reduced noise so as to be able to accurately record small nerve signals, which is a necessary prerequisite to therapeutic neuromodulation.

Inability to record microvolt-level nerve activity in the presence of stimulation pulses applied to the same nerve. During stimulation, electrode-generated stimulation artifacts often produce a shift in the differential mode signal and the common mode signal, both of which need to be properly rejected. Subtraction of the stimulation artifact requires compensating for the frequency-dependent amplitude and phase of the transfer function. This subtraction process cannot be done as a matter of routine because the process requires a very high precision amplifier buffer to store the collection of superimposed artifacts and the collected neural signals. As noted, in brain recording experiments, the amplitude of neural signals (spikes and field potentials) is seen to be around a few hundred μV, but the amplitude of stimulation artifacts can reach as great as several Volts. To deal with a signal amplitude that can be less than 10 μV, there is a requirement for higher precision electronics capable of carrying out linear amplification. Every extra bit of increased precision of this type requires a 4-fold increase in the supplied power, which is consistent with the thermal slope in commercial analog electronics. As a result of this phenomenon, a commensurate upgrade of present-day brain technologies that could support simultaneous recording and stimulation on a peripheral nerve would require a multi thousand-fold increase in power. A power source capable of such power will be relatively large, bulky, and highly undesireable from a patient's point of view, possibly even leading to patient non-compliance with use of the device.

Mismatch error. A central problem in the development of neurostimulator designs for electrical stimulation of neurons is referred to by those of ordinary skill in the art as that of mismatch error. In a signal processing system several operations or computations are performed on a signal in different stages sequentially. Each of these operations seeks to emphasize a wanted component or property of the signal of interest, without adding too much, or too many, unwanted extra components. These unwanted extra components are now known to be due to the non-idealities of a given circuit's implementation compared to the circuit's specified operation. Circuit non-idealities can be divided into two groups: random errors; and systematic errors.

In this field, random errors are the result of the randomly-determined, or stochastic, nature of many physical processes. The stochastic behavior of charge carriers in a conductor, for instance, results in various types of noise signals, and the stochastic nature of the physical phenomena that take place during the manufacturer's fabrication of integrated circuits results in a random variation of the properties of the fabricated on-chip devices and furthermore results in mismatches between identically designed devices.

Systematic errors occur because a typical circuit implementation only approximates an ideal signal processing operation to a limited extent. These errors are caused, for instance, by the non-linear operating characteristics of devices or by the influence of parasitics (unwanted resistance, capacitance, or inductance in a circuit element) in the signal path or device structure.

The effect of these non-idealities may be of different kinds. The non-ideality of noise signals limit the minimal signal that can be processed with the system. The non-ideality of device mismatch limits the accuracy of the circuit behavior and again limits the minimal signal or energy that is required to execute meaningful signal operation functions. For linear systems, the non-linearities of devices generate distortion components of the signals or modulate unwanted ‘noise’ signals into the used signal band. This typically limits the maximal signal that can be processed correctly.

The circuit designer of ordinary skill in the art has heretofore attempted to reduce the effect of such distortion non-idealities by using small modulation indices for the bias signals. By using large device sizes, the impact of mismatch is lowered and by using low impedance levels, the thermal noise signals are reduced. Such measures have, however, very important consequences on the power consumption and operational speed of the system. Therefore, the quality of a circuit realization is evaluated from the obtained accuracy, noise level, or linearity relative to the used power and the speed of operation, all in an effort to achieve, for a given speed, an optimized balance of best performance with minimal power consumption.

Transistor mismatch. In the specialized circuit case of transistor mismatch, the circuit designer of ordinary skill can reduce the effect of distortion non-idealities by the use of small modulation indices for the bias signals; so by using large device sizes, the impact of mismatch is lowered, and by using low impedance levels, the thermal noise signals are reduced. These measures have, however, very important consequences on the power consumption and operation speed of the system. Therefore, the quality of a circuit realization is evaluated from the obtained accuracy, noise level or linearity relative to the used power and the speed of operation. The designer would try to achieve, for a given speed, the best performance with minimal power consumption.

Inherent transistor mismatch. Any two identically designed devices, here transistors, on an integrated circuit have random differences in their behavior and show a certain level of random mismatch in the parameters which model their behavior. This mismatch is due to the randomly determined, or stochastic, nature of physical processes that are used to fabricate such devices. Mismatch is thus the process that causes time-independent random variations in physical quantities of identically designed devices. For example, the mismatch of two complementary symmetry metal-oxide silicon transistors (CMOS), that are identical in their manufacture, is characterized by the random variation of the difference in their threshold voltage V_{T0}, their body factor γ and their current factor β. For technologies with a minimal device size larger than typically 2 μM, a widely accepted and experimentally verified model for these random variations is a normal distribution with mean equal to zero and a variance dependent on the gate-width W and gate-length L and the mutual distance D between the devices. The characterization of transistor mismatch is a tedious process which requires a very large measurement effort. The design, realization, and validation of the measurement set-up, the acquisition of the experimental data and the statistical processing of the data have to be performed with great care to avoid errors and systematic effects. Moreover, when migrating towards sub-micron and deep-sub-micron technologies, the standard mismatch models have to be checked for their validity; if necessary, the effects of the short or narrow channel effects have to be accounted for in model extensions. A general relation can be derived for the minimal power consumption of a signal processing system due to the effect of the mismatch in the components. Transistor mismatch (as well as the phenomenon of thermal noise) are both random processes and therefore put a limit on the smallest signal that can be processed in a circuit; both phenomena will impose a minimum power consumption to achieve a certain drive, specification, accuracy, and speed. Prior art knowledge in the field of transistor mismatch held that such mismatch puts a fundamental limitation on the maximal total performance of analog signal processing systems, since the Speed-Accuracy/Power ratio is fixed by technological constants that constrain the ability to express the theoretical matching quality of the technology as a device.

Exemplary prior art efforts and attempts to control and reduce mismatch are discussed in “Implications of transistor mismatch on analog circuit design and system performance”, https://www.ee.columbia.edu/˜kinget/papers_files/chapter3.pdf, the entire disclosure of which is incorporated herein by reference, and which readily shows the great degree of challenge and importance placed on the problem of reducing mismatch. The origins of transistor mismatch, on the other hand, are linked to the given device structure, and device physics, and to the fabrication technology of integrated circuits. Device mismatch originates from the stochastic nature of physical processes used for the industrial fabrication of devices, such as ion implantations, diffusions, or etching. The device structure using a channel in a doped material and its operation by modulating the channel resistance result in random fluctuations of the device's properties and operation. For integrated circuit technologies that are fabricated and used today, these physical limitations are very fundamental and device mismatch is unavoidable. From this perspective, the limits imposed by device mismatch are restricted to signal processing systems realized using integrated circuits. As such, they are of course very important in the quest for minimal power consumption in integrated circuits. Prior art attempts to cope with the above described challenges include the following fields.

Deep neural networks to process nerve neural signals. Recording nerve neural signals with cuff electrodes is an important milestone towards developing a high-performance, minimally invasive neural interface. The thrust of this is to develop tools to analyze and understand the observed neural signal recordings. Differing from a brain single-unit recording which contains activities of a few neurons, a cuff electrode records neural signals from a nerve bundle of thousands of axons, where the recorded signals can vary in shape and in pattern, and are characterized in having poorer signal to noise ratios. Therefore, methods commonly used to process brain signals (for example, spike sorting, firing interval engineering, and rate based codings) will be less effective in processing nerve neural data. The ability to separate weak neural signals from background noise is crucial in nerve signal processing. Signal detection is preferably accomplished in a preferred embodiment of the invention by using a modified deep variational autoencoder (VAE) means for signal detection. An exemplary deep VAE consists of sequentially connected encoder and decoder networks, where the encoder learns a class label y and a probability distribution of the code z with stochastic variables of the input data x, and the decoder aims to reconstruct the input based on the class label and the code. Use of such a deep VAE means may enable the development of a large-scale, well-annotated nerve dataset, as well as a thorough exploration of inputted signals and noise, and the generation of representations of the collected and inputted signals and noise, all through the use of the deep VAE, which in turn enables the enforcement use of a de-noising criterion such that the noise will be maximally removed. After such a training phase, the deep VAE will be able to de-noise the received data in a subject or patient and hence to improve signal detection. The present invention's novel use of an improved and novel VAE is performed in combination with a novel dataset and a novel de-noising algorithm.

Datasets. Deep learning algorithms rely on large-scale, well-annotated datasets to achieve a superior performance. For example, ImageNet, a large-scale visual database designed for use in visual object recognition software research, contains over 14 million hand-annotated images, and is considered by those of ordinary skill in the art as having enabled a revolution in deep learning. The database of annotations of third-party image is freely available directly from, for example, ImageNet at https://www.image-net.org. In the present invention, to bridge deep learning and neural signal processing, a dataset similar to the taxonomy and annotation strategy of ImageNet is first constructed, according to procedures and processes well known to those of ordinary skill in the artificial intelligence (AI) arts. The dataset then is used for developing neural signal processing algorithms.

Dataset generation. Cuff electrode data are a data summation from both filtered intraneural signals (signals within the nerve) and from noise arising out of external sources. Normally, cuff electrode data (both signals and labels) are not available for learning algorithms, especially for supervised learning, to record high quality, multi-site intraneural signals, and must be generated by the user or practitioner as part of the practice of the invention as claimed. The inventors have now built a cuff electrode dataset based on intraneural signals. First, a finite element model of epineurium signals is developed to simulate cuff electrode neural signals based on multi-site intraneural signals. To this database is then added noise that has been segmented from cuff electrode recordings, and the procedure is repeated with data from different electrodes and data from experimentally-derived and from third party-sourced animal model preparations. This yields a dataset derived from a sufficient set of experiments that the dataset can support the development of supervised learning algorithms to process neural signals.

Deep learning de-noising. Mathematically, the data representation process with the deep VAE can be expressed as:

pθ(x,y,z)=pθ(x|y,z)p(y)p(z)

where pθ(x|y, z) quantifies how the observed values of x are related to the latent random variables y and z, and p(y), p(z) represent a known prior distribution of the latent variables y and z. Given this representation model, the posterior distribution pθ(y|z, x) can be used to infer y, z and to find parameters θ that maximize the marginal likelihood pθ(x). To approximate the intractable pθ(y|z, x) a decoding distribution qΦ(y|z, x) is modeled by learning the parameters Φ from the data. Next, one considers a 1-D time-series input xₜ={Xₜ−T1, . . . , Xₜ+T2}, where X represents a single-electrode recording and [t−T₁, t+T₂] is a temporal scanning window. The binary classification label (i.e., either neural signals or noise) at time t is denoted as a one-hot vector yₜ, and the corresponding latent variables are represented as zₜ. The preferred embodiment of the deep VAE of the present invention models a joint distribution according to pθ(xₜ|yₜ, zₜ) factorized as pθ(xₜ, yₜ, zₜ)=pθ(xₜ|yₜ, zₜ)p(yₜ)p(zₜ). For the decoder model, the present invention uses pθ(xₜ|zₜ, yₜ)=N(μθ(zₜ, yₜ), σ²θ(zₜ, yₜ)I); and for the encoder model, relying on the theory of variational inference to approximate the intractable posterior, pθ((z|x, yₜ) with a tractable auxiliary distribution qΦ(zₜ|xₜ, yₜ)=N(μΦ(xₜ, yₜ), σ²Φ(xₜ)I). In the supervised case with an annotated dataset, the label yₜ is observed, allowing the parameters θ and Φ to be optimized by maximizing the extended variational lower bound as expressed as:

log p_{θ}(xₜ,yₜ)≥E_{qΦ}(zₜ≥|xₜ,yₜ)[log p_{θ}(xₜ|yₜ,zₜ)+log p_{θ}(yₜ)+log p_{θ}(zₜ)−log q_{Φ}(zₜ|xₜ,yₜ)]=def L_{L}(xₜ,yₜ).

For de-noising a nerve recording, the user injects the cuff electrode noise ε into xₜ to synthesize cuff recordings ^{˜}xₜ=xₜ+ε, where the noise is picked from the cuff data.

Simultaneous recording and stimulation on a peripheral nerve. Proof of concept of electroceuticals requires integrating the stimulation function and performing personalized adaptive neural modulation therapies based on neural feedback. Another challenge that the invention had to overcome is that nerve neural signals are relatively very weak, and thus they are vulnerable to the stimulation noise artifacts. To reduce such noise artifacts, a key feature of the novel invention as claimed is that of the novel redundant crossfire (RXF) stimulator design, based upon redundant sensing scientific theory.

Redundant sensing. Redundancy is a fundamental characteristic of many biological processes such as those in the genetic, visual, muscular, and nervous systems; yet its underlying causative driving mechanism is not well understood. A complete discussion of the phenomenon of redundancy is set forth at A Bio-inspired Redundant Sensing Architecture, accessible at https://papers.nips.cc/paper/6564-a-bio-inspires-redundant-sensing-architecture.pdf the entire disclosure of which is incorporated herein by reference. The present invention utilizes the redundancy inherent in materials engineering to enhance the accuracy and precision aspects of the system, by focusing on the application of the phenomenon of redundancy to reduce stimulation signal residual charge, and thus reduce the effects of stimulation noise artifacts. In the invention's use of redundant sensing, each entry of information can be represented by a plurality of distinct configurations or microstates, and there is a distinct subset of such microstates that will allow linear representation of the entries of information, and such a set or subset will not be bounded by the classic Shannon limit (see C. E. Shannon, The Mathematical Theory of Communication, by Claude E. Shannon and Warren Weaver. University of Illinois Press, 1964, the entire disclosure of which is incorporated herein by reference) when processed according to the practice of the present invention. The invention's identification of an optimized subset is an NP-incomplete problem, but it is possible to find a sub-optimal solution with sufficient efficiency to obtain a well-operating final complete embodiment of the invention. For example, in the case where a target stimulus signal is a 100 μA biphasic current, with a 6-bit resolution in amplitude, the anodic and cathodic branches will have up to a 3% mismatch depending on electrode conditions and clock jitter. Thus, the mismatch current is 100 μA×(3%+½⁶)=4.5 μA, which represents the cause of the resulting residual charge and stimulation noise artifacts. It can be seen from this example that a given amount of mismatch is stimulus dependent, time variant, and sensitive to electrode-electrolyte offset, thus posing a significant challenge to effective reduction of residual charge and stimulation noise artifacts, without having to resort to the prior art strategies of increasing the amount of power consumed or increasing the size of the neurostimulation device, both of which are product design strategies that ultimately produce a finished device of poor ergonomics and disappointing user satisfaction, possibly even resulting in patient noncompliance.

RXR stimulator. Based on the redundant sensing strategy, the present invention comprises an redundant crossfire, or RXF, stimulator, wherein the outputs of two or more independent stimulation channels with a current-digital-to-analog converter (IDAC) output driver will effectively form a redundant sensing structure. The sensed redundancy is exploited to fine tune and achieve precise matching between the anodic and cathodic stimulation currents, thus suppressing the residual charge and stimulation noise artifacts.

Accordingly, a need exists for a solution to at least one of the aforementioned challenges. The present invention addresses the challenges of the shortcomings of the prior art in attempting to solve the problems in circuit engineering described above, by designing a high-resolution constant-current stimulator (CCS) in integrated circuits without incurring penalties such as relatively large silicon area and/or high degree of power consumption because of the phenomenon of electronic component mismatch error in general, and transistor mismatch error in particular. Studies have shown that the phenomenon of random mismatch error is one of the major factors in limiting circuit design's effective resolution of such problems, especially when scaling down the circuits to submicron CMOS processes. Arising from random variations in the lithography process that are beyond the designers' control, mismatch error is the scourge of high-resolution analog IC designs. Nearly all neurostimulator designs are affected because mismatches appear in any type of component on an integrated circuit (IC) such as transistors, diodes, resistors, and capacitors. Current-controlled stimulators (CCS) are primarily affected by transistor mismatches, while voltage-controlled stimulators (VCS) and switched-capacitor stimulators (SCS) are primarily affected by transistor and capacitor mismatches. Mismatches influence the circuit's ability to produce accurate current/voltage output and synchronously match negative and positive stimulation phases, leading to charge imbalance problems. The work and resources involved in compensating for these errors requires extra calibration circuits, which in turn add complexity, area, and power.

Exploitation of the phenomenon of excessively large mismatches. All previous works in the literature have employed calibration circuits or chip layout techniques to attempt to remove or compensate for random mismatch error. Here, in the present invention as claimed, we disclose an entirely different approach to address this challenge, not by avoiding or compensating the mismatch error, but by embracing and exploiting excessively large mismatches to achieve super-resolution over 10-fold beyond the intrinsic resolution of the design. The redundant crossfire, or RXF, circuitry and method involves combining (i.e., crossfiring) the output of two or more current drivers to form a redundant structure. In the presence of large random mismatch errors (10-20%), such resultant redundant structure may be configured to generate the output that has an effective resolution that is many times above the Shannon limit as determined by physical constraints, including the number of unit-transistors. RXF in the present invention as claimed is a practical derivation of the theoretical framework that includes RS and RS-based super-resolution, wherein information redundancy is elegantly engineered and manipulated to boost system resolution. Furthermore, by handling extremely large mismatches, the present invention now makes it possible to implement high-resolution analog circuits in deep submicron technologies.

High-resolution, fully-integrated neurostimulators are at the core of many implantable or wearable devices. The neurostimulator's function is to generate stimulation pulses with accurately controlled parameters such as pulse-width, current amplitude, and frequency. It is a well-established method for interfacing with neural circuits well known to those of ordinary skill in the art. Proper uses of neurostimulation potentially offer a viable conduit to relay information to the human brain, and neurostimulation is widely used in neuroprosthesis applications to augment or replace missing or degraded human sensory functionality. Uses can include retinal implants for restoring vision, cochlear implants for restoring hearing, and prosthetic limbs for restoring somatosensation.

The present invention demonstrates a specific, most highly preferred application of a novel neurostimulator chip, in a prosthetic limb neuroprosthesis system, for the restoration of somatosensation for an amputee. A similar design can be adopted in a wide range of neuromodulation applications where the stimulator is the essential component of a bidirectional human-machine interface.

In a preferred embodiment of the present invention, a novel circuit, conceived of to be a component of one or more integrated circuits, is employed in order to utilize a redundant crossfire (RXF) technique in the design of neurostimulators, with super-resolution accuracy. The technique derives from the redundant sensing circuit architecture known to those of ordinary skill in the art. See Luu et al. (2019). Achieving Super-Resolution with Redundant Sensing. IEEE Transactions on Biomedical Engineering, 66(8), 2200-2209. (2019), the entire disclosure of which is incorporated herein by reference. There, the aim was to achieve engineering information redundancy, built into the system's architecture, in order to exploit the phenomenon of random transistor mismatch, and to thereby enhance the overall effective resolution capability of the device. The application of RXF in the present invention involves combining the RXF (i.e., crossfiring) outputs of two or more current drivers in order to form a redundant structure. See Nguyen et al. (2021). Super-Resolution in Neurostimulator Design by Exploiting Transistor Mismatch. IEEE Journal of Solid-State Circuits, 56(8), 2452-2465. (August 2021), the entire disclosure of which is incorporated herein by reference. When properly configured, this novel redundant structure can produce accurate current pulses with an effective super-resolution that is beyond the limitation commonly permitted by the physical constraints. Notably, and unlike any previous works, the novel circuitry of the device of the present invention unexpectedly and surprisingly achieves high accuracy by directly exploiting random transistor mismatch with an excessively large mismatch ratio of 10-20%, an outcome which cannot have been predicted with a reasonable degree of success.

In one most preferred embodiment of the present invention, effectiveness is significantly increased by, for example, incorporating a neurostimulator chip characterized in that each of a plurality of current drivers has a 5-bit current digital-to-analog converter (IDAC) and two 4-bit current multipliers. By crossfiring two drivers, it is feasible to achieve an effective super-resolution of 9.75 bits in a 1.1 mA full-range or 27× the ideal intrinsic resolution of each IDAC. The performance boost is verified through benchtop measurements of fabricated chips, and is consistent with the Monte Carlo (i.e. a broad class of computational algorithms that rely on repeated random sampling to obtain numerical results, where the underlying concept is to use randomness to solve problems that might be deterministic in principle) simulations that were run, demonstrating that the proposed technique is robust and dependable. A most highly preferred application of the fabricated chip here is to deliver neuro-feedback to a human amputee through peripheral nerves, where the amplitude of stimulation pulses is being accurately controlled in order to encode the intensity of tactile response.

The process of quantization i.e., analog-to-digital conversion (ADC) and the reverse operation de-quantization i.e., digital-to-analog conversion (DAC) are the basis of all modern sensory data acquisition systems. They allow “digital” artificial systems to sense and interact with the “analog” physical world. Quantization is essentially a lossy data compression process where information from a higher-resolution space is represented in lower-resolution counterparts. In practical implementations, the precision of this process is always bounded by the system resource constraints such as size, power, bandwidth, and memory, etc. For example, in many ADC and DAC integrated circuits designs, an addition 1-bit of resolution or 2× precision often require a 4× increase of chip area and power consumption. While ultra-high resolution ADCs/DACs up to 32-bits are possible, the large size and power consumption limit the use of these devices in many practical applications. Similarly, higher resolution image sensor requires more pixel count and buffer memory thus also results in larger device and power consumption. While it is possible to improve the pixel density, the smaller pixel size is associated with increased noise which limits the sensor's dynamic range.

Super-resolution (SR) are techniques that aim at achieving an effective resolution exceeding the precision that the system's resource constraints commonly permit. They have wide applications in various fields of engineering and science concerning imaging and instrumentation where higher resolution data acquisition is always desired. Previous SR techniques focus on recovering fine details of the object of interest by integrating the information obtained from coarse observations. These techniques could be generally divided into two primary classes: modeling-based and oversampling-based, which are also known as single-frame and multi-frame in image processing.

Modeling-based (single-frame) techniques focus on modeling the input sources from available data points and reconstructing the missing information by means of approximation. In these techniques, SR is achieved by relying on known statistical properties of the input signals such as their sparse property as utilized in compressive sensing, or properties extracted from numerous example data as utilized in many machine learning based methods. On the other hand, oversampling-based (multi-frame) techniques acquire and combine multiple samples of the input obtained at various spatial or temporal instants to extract the sub-least-significant-change information. In these techniques, SR is possible because the low-resolution data contain aliasing which embeds high-resolution contents that can be extracted with sufficient amount of data by algorithm based (e.g., de-noising, deconvolution, etc.) or machine learning based methods. For compressive sensing or other data-driven methods including most existing machine learning based techniques, optimization or approximation is performed during the reconstruction process, after the low-resolution data have been acquired.

Additionally, mismatch error is one of the major obstacles hindering the implementation of high-precision DACs/AD Cs in sub micron CMOS processes. Mismatch error is the random deviation taken place in during the fabrication of integrated circuits (ICs). Mismatch error can result in random changes of the intrinsic properties of the IC components including active electrical elements such as transistors and/or passive electrical elements such as capacitors and/or resistors, which leads to unpredictable behavior of the circuit and the degradation of the overall system precision. For example, a DAC/ADC designed with 10-bit resolution may only have an effective resolution about 8-9 bits in practice due to mismatch error.

It would therefore be desirable to provide systems and methods for designing digital-to-analog converters that provide super-resolution without post-processing and in presence of mismatch error.

### SUMMARY OF THE INVENTION

In summary, the invention, in its most preferred embodiment, is a neurostimulator system, comprising at least one digital-to-analog converter configured to receive an analog peripheral nervous system electrical signal from a subject or a patient, and to convert the analog signal into a corresponding digital signal; at least two current mirror circuits configured to receive digital electrical signals from the digital-to-analog converter and to provide mirrored current to at least two additional circuit components; at least two or more current drivers, at least one being an anodic output current driver, and at least one being a cathodic output current driver, the drivers being configured to scale the current signals received from the mirror circuits by a multiplying factor, and further configured to driving the constant current to at least one output electrode; wherein the outputs of the two or more current drivers are configured so as to create a combined, crossfiring, output of the current drivers that produces a redundant sensing structure, that in turn produces accurate current pulses that have the property of having an effective super-resolution accuracy beyond the ordinary limitations that are imposed by the predicted inherent physical constraints of materials in the system. The redundant sensing structure is configured so as to achieve a super-resolution signal accuracy outcome by applying the effects of random mismatch error function to the system, with the proviso that mismatch avoidance and mismatch compensation functions are not applied in achieving such super-resolution signal accuracy outcomes. In a most highly preferred embodiment, the random mismatch error function is configured so as to select and tune transistor size to achieve a desired mismatch ratio of 10% to 20%. Preferably, the system additionally comprises an on-chip timing generator, and additionally comprises both on-chip and off-chip components that are each configured so as to retrievably store calculated optimal transistor configurations obtained through foreground calibration, and which configurations can be retrieved and read by the on-chip timing generator so as to produce signal output with super resolution accuracy. Such an on-chip component is preferably a memory chip component of the system, whereas the off-chip component is a look-up table component of the system. The system additionally comprises an external controller configured so as to ensure charge-balancing that is achieved by digital compensation for residual mismatch between the anodic and cathodic currents. This charge balancing capability of the system is further characterized as being able to finely level charge balancing within the system, or to coarsely level charge balancing within the system. The system is configured so as to modulate the neurostimulation intensity of the cross firing redundant signal output to create various levels of somatosensorial signal outputs of from light to strong touch in real time in a neuroprosthesis device. The described random mismatch error function is configured so as to select and tune resistor size or to tune capacitor size or to tune diode size to achieve a desired mismatch ratio. A preferred embodiment of the system is configured so as to apply the effects of a random mismatch error function in the system, in turn configured so as to be applied to extremely large mismatch sets to achieve super-resolution that is over 10-fold beyond the intrinsic resolution of the system that one of ordinary skill would predict as being imposed by the physical constraints of the materials utilized in the manufacture of components in the system. The circuit of the invention may be embodied in a high-resolution constant-current stimulator neuroprosthesis neurostimulator chip. The circuit of the invention may be embodied in an electrical neuromodulation neurostimulator chip for the generation of neurostimulation signals in a neuroprosthesis. The invention additionally comprises a method of rehabilitating an amputee by fitting the amputee with a tactile-sensitive neuroprosthesis comprising the neurostimulator chip of the present invention. A preferred neuroprosthesis is a prosthetic forearm and hand while an alternative embodiment preferred neuroprosthesis is a prosthetic hand.

In another aspect, the incorporation of, and combination of, a digital-to-analog converter device is provided by the present disclosure. The digital-to-analog converter device includes a set of components, where each component included in the set of components includes a number of unit cells and at least one component including a number of unit cells that is not a power of two. The digital-to-analog converter device further includes a plurality of switches, each switch included in the plurality of switches being coupled to a component included in the set of components, an output electrode coupled to the plurality of switches, the digital-to-analog converter device being configured to output an output signal at the output electrode, and a controller coupled to the plurality of switches. The controller is configured to receive a desired output current, determine an anodic component configuration including at least one component included in the set of components based on the desired output current, determine a cathodic component configuration including at least one component included in the set of components based on the desired output current, and cause a current pulse to be output at the output electrode based on the anodic component configuration and the cathodic component configuration.

In the digital-to-analog converter device, the current pulse can include a positive current pulse and a negative current pulse.

In the digital-to-analog converter device, the controller can include a memory that can include a set of positive current values and negative current values associated with a set of component configurations, and the anodic component configuration and the cathodic component configuration can be included in the set of component configurations.

In the digital-to-analog converter device, the anodic component configuration can include at least one component not included in the cathodic component configuration.

In the digital-to-analog converter device, an effective resolution of the digital-to-analog converter device can be at least four times greater than an intrinsic resolution of the digital-to-analog converter device. The effective resolution of the digital-to-analog converter device can be equal to a Shannon entropy of the digital-to-analog converter device, and the intrinsic resolution can be equal to log base two of the number of unit cells plus one.

The digital-to-analog converter device can be included in a neurostimulator device.

In the digital-to-analog converter device, a first unit cell size associated with a first unit cell included in the set of components can be different than a second unit cell size associated with a second unit cell included in the set of components, and the first unit cell size can include a length and width of the first unit cell. The first unit cell size and the second unit cell size can be associated with a transistor process size.

In the digital-to-analog converter device, each unit cell can include at least one transistor.

In the digital-to-analog converter device, an effective resolution of the digital-to-analog converter device can be at least two hundred times greater than an intrinsic resolution of the digital-to-analog converter device for at least ninety-five percent of a sample space of the digital-to-analog converter device.

In another aspect, a digital-to-analog converter device including a set of components, each component included in the set of components including a number of unit cells, each unit cell being associated with a unit cell size indicating manufacturing specifications of the unit cell is provided by the present disclosure. The digital-to-analog converter device further includes a plurality of switches, each switch included in the plurality of switches being coupled to a component included in the set of components, and an output electrode coupled to the plurality of switches. The digital-to-analog converter device is configured to output an output signal at the output electrode. A first unit cell size associated with a first unit cell included in the set of components is different than a second unit cell size associated with a second unit cell included in the set of components.

In the digital-to-analog converter device, the unit cell size can include a length value and a width value.

In the digital-to-analog converter device, the unit cell size can be associated with a transistor process size.

In the digital-to-analog converter device, at least one component included in the set of components can include a number of unit cells that is not a power of two. [0022] The digital-to-analog converter device can be a current digital-to-analog converter.

The digital-to-analog converter device can further include a controller coupled to the plurality of switches. The controller can be configured to receive a desired output current, determine a component configuration based on the desired output current and a predetermined output current value measured at the output electrode, the predetermined output current value associated with the component configuration, and cause a current pulse to be output from the digital-to-analog converter device based on the component configuration.

In yet another aspect, a method for determining manufacturing parameters for a digital-to-analog converter device including a set of components, each component included in the set of components including at least one unit cell, and each unit cell being associated with a unit cell size is provided by the present disclosure. The method includes determining a required mismatch error value for the unit cells included in the component set based on a targeted effective resolution value, determining an initial unit cell size based on the required mismatch error value, setting the unit cell size of each unit cell included in the component set to be equal to the initial unit cell size, determining an effective resolution of the digital-to-analog converter device by performing simulations, determining that the effective resolution is below the targeted effective resolution, adjusting the unit cell size of one or more unit cells included in the component set in response to determining that the effective resolution is below the targeted effective resolution, and providing each unit cell size associated with each unit cell to a manufacturing facility.

In the method, the targeted effective resolution can be at least four times higher than an intrinsic resolution of the digital-to-analog converter device.

In the method, the unit cell size can include a length value and a width value, and each unit cell can include at least one transistor.

In the method, at least one component included in the set of components can include a number of unit cells that is not a power of two.

In the method, the simulations can be Monte Carlo simulations.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A and 1B are circuit diagrams that provide circuit schematics that show the basic reverse crossfire, or RXR, circuit components of the invention.

FIG. 2 is a four-part circuit diagram that provides circuit schematics of four alternative embodiments that achieve an identical neural signal output.

FIG. 3 is as distribution chart displaying the number of redundant configurations corresponding to individual output codes of various RXF structures.

FIGS. 4A, 4B, and 4C are graphical representations of varying degrees of configuration code diffusion.

FIGS. 5A and 5B are the graphical results of a Monte Carlo simulation of super-resolution created by the invention.

FIG. 6 is the graphical result of a Monte Carlo simulation of random mismatch ratio to achieve super-resolution.

FIG. 7 is a circuit diagram that provides a circuit schematic of a fully integrated neurostimulator design.

FIG. 8 is a tabular chart of typical physical measurements of transistors used in the RXF circuit.

FIG. 9 is a schematic flow-chart of the process of tuning transistor mismatch.

FIG. 10 is the graphical results of a Monte Carlo simulation of the effect of tuning on effective mismatch.

FIG. 11 is a flow chart and schematic illustration of a stimulator chip control strategy.

FIG. 12 is a micrograph of a neuromodulator chip of the invention.

FIGS. 13A and 13B are graphical representations of chip performance under varying mismatch management strategies.

FIG. 14 is a graphical representation of actual computed mismatch error under varying mismatch management strategies.

FIGS. 15A and 15B are graphical representations of a stimulator channel integral nonlinearity and of differential nonlinearity.

FIGS. 16A and 16B are graphical representations of the results of output measurements that demonstrate that decreasing current levels yield lowered distortion levels.

FIG. 17 is a graphical representation of charge mismatch as a function of charge-balancing capacity of a stimulator channel.

FIG. 18 is a graphical representation of the results of a stimulator's charge-balancing characteristics measured in a buffered saline solution.

FIG. 19 is a set of photo images of a prosthesis incorporating a neurostimulation module as part of the prosthesis complete system.

FIG. 20 is a graphic representation readout of current amplitude pulses generated by prosthesis tactile contact with a solid surface.

FIGS. 21A and 21B are a pair of graphic representations of before-and-after nerve signal quality following stimulation artifact removal.

### DETAILED DESCRIPTION OF THE DRAWINGS AND INVENTION

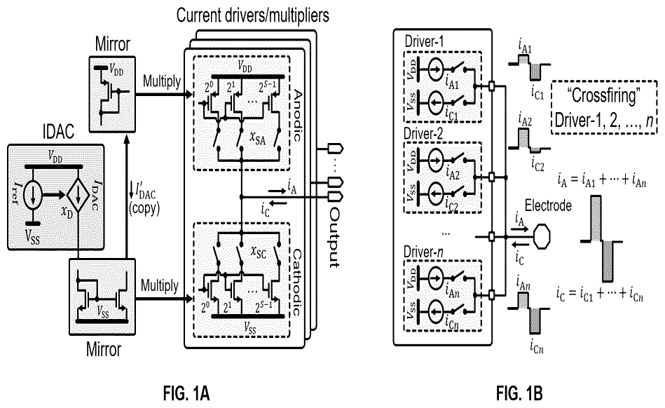

Turning first to FIGS. 1A and 1B, there is shown a preferred embodiment of the basic RXF circuitry components, so configured as to create a structure of information redundancy, it being understood herein that numerous alternative different internal configurations can produce the same type of desired output. An important consideration is that a circuit architecture as disclosed here will realize its desired output qualities with a significantly reduced requirement of physical components, as compared to the prior art.

FIG. 1A illustrates a preferred embodiment of a standard, biphasic, current-mode stimulator, which is comprised of a current digital-to-analog converter (IDAC) 101, current mirror circuits 102 and 103, and anodic (positive) 104, and cathodic (negative) 105 output current driver/multipliers. The drivers perform dual functions: firstly, the scaling of the IDAC current by a multiplying factor, and secondly, the driving of a constant current to an electrode. For each channel of a given embodiment, there is one IDAC that is shared among a plurality of discrete drivers. Subsequently, the anodic i_{A} and cathodic i_{C} output currents are expressed as follows:

i_{A}=I_{IDAC}·x_{SA}=I_{ref}·x_{D}·x_{SA}

i_{C}=I_{IDAC}·x_{SC}=I_{ref}·x_{D}·x_{SC}

Where I_{ref} is a fixed reference current, x_{SA} and x_{SC} are the multiplier codes, and x_{D} is the IDAC code.

FIG. 1B illustrates how an RXF structure is created by functionally connecting and combining, or as used in this disclosure, “cross firing” the output of two or more drivers 106, 107, and 108. Each driver contributes a small amount of current shown as pulse forms 109 for driver 1, 110 for driver 2, and 111 for driver n, which together constitute the input current i_{A} 112, which may be independently adjusted to generate a final desired stimulation pulse 113, and that constitutes the output current i_{C} 114. The timing of the pulses produced by different drivers must be synchronized, meaning that the anodic and cathodic currents are turned on and off at exact moments in time such that they will behave as a single stimulation channel. This synchronization can be easily achieved with an on-chip timing generator means, well known to those of ordinary skill in the art, that turns on, and turns off, the output from two or more drivers simultaneously at desired and calculated time internals so as to achieve synchronization of the outputs.

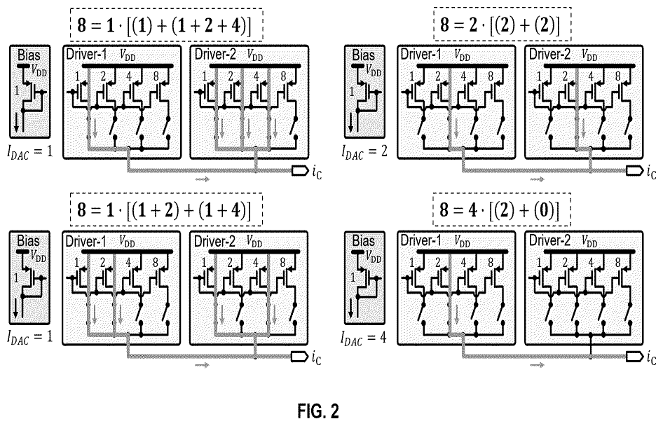

FIG. 2 illustrates examples of several alternative circuit configurations that produce the same theoretical output when there is no mismatch error. Here the IDAC's resolution is expressed as N_{D}=5 and the multiplier's resolution is expressed as N_{S}=4. These four illustrated configurations of different IDAC and multiplier values will generate the same output code i_{C}=8. The RXF structure is seen to be redundant because the same output code can be generated by numerous different configurations of the IDAC and multiplier. In practice, there may be up to tens to hundreds of distinct configurations associated with each output code. The varying configurations of the IDAC and multipliers exhibit complex mutual relationships that depend on the resolution capability of the IDAC, of the multipliers, and of the number of crossfire drivers.

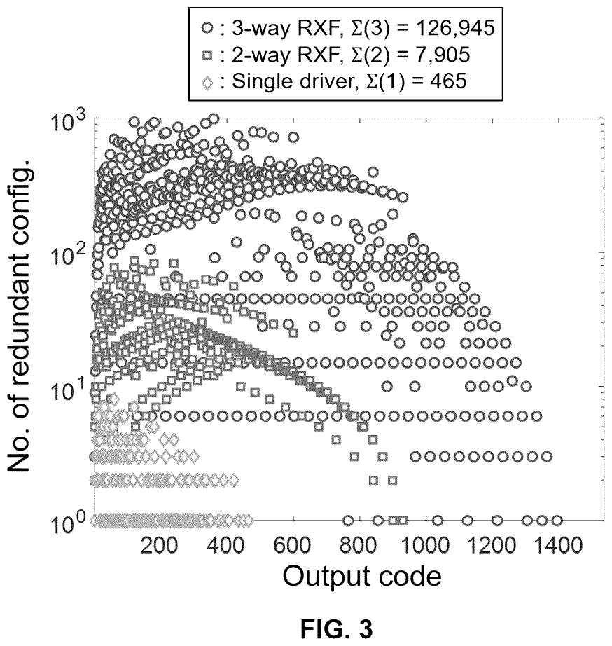

FIG. 3 illustrates a distribution of the number of redundant configurations 301 corresponding to each output code in a single driver, in this non-limiting example given as a 2-way RXF 302, and a 3-way RXF 303 structure across the sample space, compared to an apparatus having a single driver 304. Manufacturing additional crossfire drivers thus can be seen to extend the maximal output, and exponentially increase the level of redundancy, which is essential to achieve super-resolution. The total number of non-zero, distinct configurations in an n-way RXF structure with n crossfire drivers is expressed as follows:

Σ(n)=(2^{N}^{_{D}}−1)·[(2^{N}^{_{S}})ⁿ−1]

With each additionally added crossfire driver, the number of configurations (i.e., level of redundancy) grows exponentially, but the physical resources required, i.e., for example chip area, will only increase linearly. This is a significant improvement in improving signal output and quality without a parallel increase in apparatus resource, which would otherwise impose physical size constraints and energy consumption constraints on a neuromodulation device.

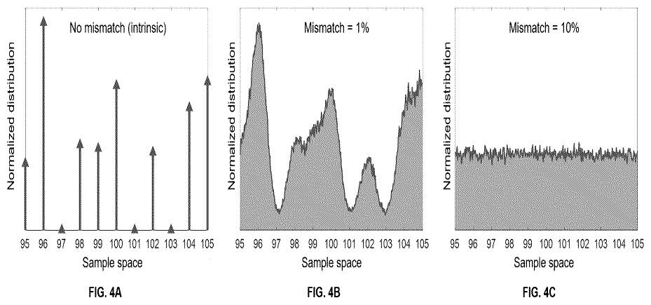

FIGS. 4A, 4B, 4C show a unique property of a redundant structure called “code diffusion” that enables super-resolution. The graphs show the distribution of the analog outputs produced by three different configurations of a 2-way RXF structure. The analog outputs are contained in a continuous sample space with values ranging from 0 to a maximum value of approximately 31·(15+15)=930 LSB where 1 LSB=I_{ref}. A least significant bit (LSB) is the bit position in a binary integer giving the units value, that is, determining whether the number is even or odd. The LSB is sometimes referred to as the low-order bit or right-most bit, due to the convention in positional notation of writing less significant digits further to the right. It is analogous to the least significant digit of a decimal integer, which is the digit in the ones (right-most) position. To model the random mismatch error, it is assumed that each unit-transistor of both the IDAC and the multipliers has a Gaussian distribution, and that the standard deviation is the mismatch ratio. When generating random samples, negative values are set to zero. FIG. 4A shows that, with no mismatch errors, redundant configurations generate the exact same analog outputs 401 that are centered at integral integer codes 402, e.g. [0, 1, 2, 3, . . . ]. Their distributions are represented by Dirac delta impulses with the weight equal to the number of configurations. FIG. 4B shows that, with small random mismatch errors, the actual values of different analog outputs begin to deviate from their original states and “diffuse” into the adjacent sample space 403. FIG. 4C shows that, with large mismatch errors, the actual values of different analog outputs have exponentially increased and completely diffused 404, and are now evenly distributed across the sample space.

Code diffusion allows for the generation of sub-integer codes, e.g. [0.1, 0.2, 0.3, . . . ] with a certain probability that is not normally possible. These sub-integer codes correspond to the sample space's finer partitions, so that there is thus an effective super-resolution that is beyond the baseline figure. For example, to achieve a (+1) super-resolution, redundant configurations that generate all the e.g. sub-integer codes of [0, 0.5, 1, 1.5, . . . ] must be found. To achieve (+2) super-resolution, the required sub-integer codes are [0, 0.25, 0.5, 0.75, 1, . . . ]. While identifying the correct configuration for every output code is an NP-hard optimization problem, it is only possible in an information redundant architecture such as RS. In computational complexity theory, a problem is NP-complete when: a brute-force search algorithm can solve it, and the correctness of each solution can be verified quickly, and the problem can be used to simulate any other problem with similar solvability; where RS is Reed-Solomon codes, which operate on a block of data treated as a set of finite field elements called symbols. RS codes are able to detect and correct multiple symbol errors. The probability of accomplishing this task is maximized when the codes distribute evenly across the sample space, as shown in FIG. 4C.

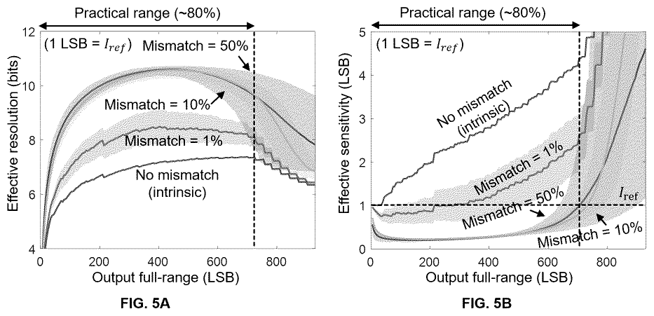

FIGS. 5A, and 5B illustrate Monte Carlo simulation results to evaluate the theoretical super-resolution that can be achieved with the present invention. Here, the mismatch error is applied to elements of both the IDAC and multiplier equally. In each simulation, the RXF structure is optimized by an automated brute-force approach, i.e., sorting through all the possible redundant configurations and finding the ones that generate the desired output with the least amount of error. We use the terms “effective resolution” and “effective sensitivity” to measure quantitative performance. The effective resolution is defined as the Shannon entropy, which is computed with respect to a targeted resolution (12 bits) is expressed as follows:

$${M}_{{N}_{x}} = {\sum }_{d = 0}^{{2}^{{N}_{x}} - 1} {\int }_{{θ}_{d}}^{{θ}_{d + 1}} {( {x}_{A} - \frac{d + 0.5}{{2}^{{N}_{x}}} )}^{2} d {x}_{A}$$

and

H_{N}_{ₓ}=log₂\sqrt{12·M_{N}_{ₓ}}

where H_{N}_{ₓ} is the effective resolution (entropy) with respect to the targeted resolution Nₓ; M_{N}_{ₓ} is the normalized total mean-square-error integrated over each digital code d∈[0, 2^{N}^{ₓ}−1]; θ₀, θ₁, . . . are the corresponding analog outputs. The targeted resolution Nₓ is the reference upper-bound of the device's super-resolution, which is arbitrarily defined over the full-range. No matter how high the targeted resolution is defined, the effective resolution H_{N}_{ₓ} would converge to a maximum value. The effective sensitivity is defined as the smallest change in output current that could be accurately produced by the device. It is computed as follows:

$${S}_{{N}_{x}} = {I}_{r e f} \cdot \frac{F R}{{2}^{{H}_{{N}_{x}}}}$$

where S_{N}_{ₓ} is the effective sensitivity; FR is the targeted output full-range. The metrics are evaluated for all values of the full-range from 0 to the maximum value of 930LSB.

FIG. 5A shows that RXF enables the achievement of an effective super-resolution that is practically always higher than the device's intrinsic resolution. The effective super resolution, at different values of output full-range, is highly correlated with the number of redundant configurations. The effective resolution in the first 0-50 LSB is low because there are not enough redundant configurations. The effective resolution reaches its highest point at approximately 50-80% (450-750 LSB) of the maximum full-range in which most of the redundant configurations are located. At the practical full-range of 80% (750LSB), the RXF structure achieves 2-3 bits super-resolution beyond the intrinsic baseline. The intrinsic baseline is the best resolution that can be attained with the prior art conventional structure and zero mismatches. Beyond this point, the resolution drops rapidly because the redundant configurations become sparsely distributed, thus there is insufficient redundancy. Moreover, the illustrated results clearly demonstrate a novel, unobvious, unique, and fundamental functional capability of the present invention, which is that mismatch error is utilized to actually enhance resolution. Unlike any previous design in the prior art, the proposed system becomes more accurate as and when the mismatch ratio increases from 0 to 10%. This is because a larger mismatch error leads to a more even distribution of redundant values across the sample space, which maximizes the probability of finding a configuration to generate the desired output. The effectiveness also becomes more consistent (i.e., smaller deviation) when the mismatch ratio increases from 0 to 10%. This shows that the super-resolution achieved by the present invention is high-yield, replicable, and does not rely on a specific random configuration.

FIG. 5B illustrates that similar functions of the invention are produced, in terms of the other parameter being used, namely effective sensitivity, which is proportional to the inverse of the effective resolution.

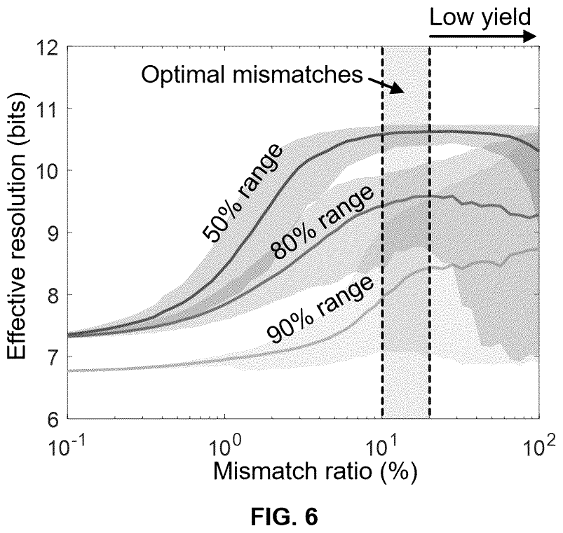

FIG. 6 illustrates Monte Carlo simulation results from evaluating the optimal range of random mismatch ratio to achieve super-resolution. The graph shows the effective resolution, computed at 50%, then 80%, then 90% ranges with different mismatch ratios of from 0.1-100%. The present invention demonstrates that in a novel, non-predicted, and surprising manner, contrary to the teachings of the prior art, a mismatch ratio within a range of about 10 to 20% yields the most preferred, optimal, super-resolution at most ranges. Above that range, not only is there is little significant additional boost to the effective resolution, but also, deviation starts to widen. This suggests a lower effective yield, since fewer samples are capable of achieving the desired super-resolution, and the structure may well become unreliable.

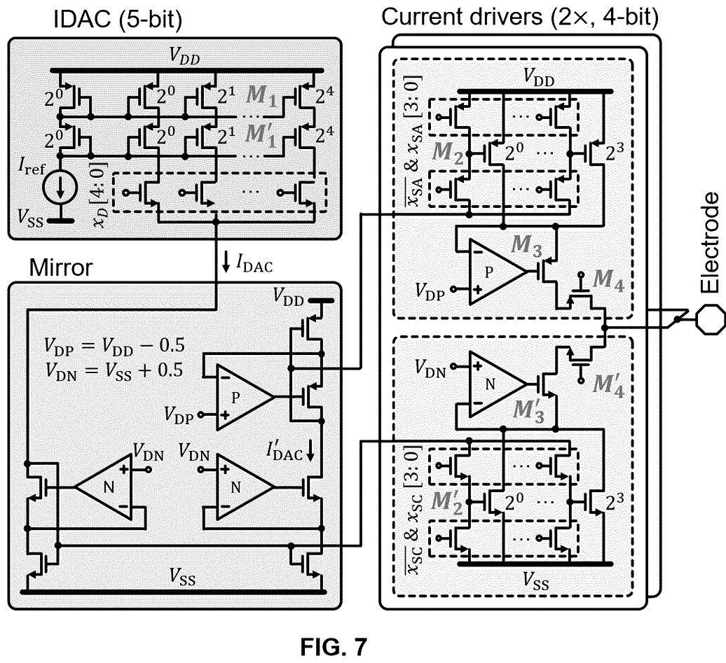

FIG. 7 illustrates the schematic of a fully-integrated neurostimulator design with 2-way RXF architecture n=2, N_{D}=5, N_{S}=4. The IDAC is shown at 701, the current mirror is at 702, and current drivers are at 703 and 704. The current mirrors and drivers are based on an op-amp assisted boosted-cascode current driver and current mirror. Here the design trades away or trades off device area in order to to achieve ultra-high-output impedance. The estimated output impedances are >1 GΩ at 1 mA (source) and >50 GΩ at −1 mA (sink). The output voltage range is set by V_{DN}=V_{SS}+0.5 (V) and V_{AP}=V_{DD}−0.5 (V), which results in compliance of ±4.5V. The reference current is generated by a voltage-to-current converter circuit, and the value is set to. I_{ref}=1.5 μA by V_{DD} and by an external resistor.

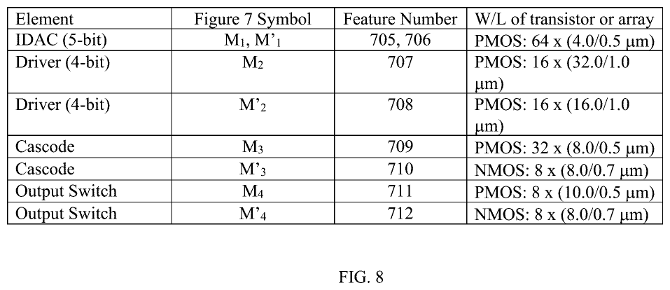

FIG. 8 shows, with respect to the circuit in FIG. 7, the width to length dimensions, in μm, of key transistors and transistor arrays illustrated in FIG. 7. Minimum feature-size transistors are used whenever possible (e.g., IDAC) to maximize the available mismatch error. Nevertheless, larger transistors are needed for the driver, the cascode, and the output switches, in order to meet the voltage-drop requirement for maintaining output compliance. The anodic circuits generally use larger transistors than their cathodic counterparts because the current drivability of P-channel metal-oxide-semiconductor (PMOS) is about 50% less than that of N-channel metal-oxide semiconductor (NMOS).

Proceeding contrary to conventional wisdom, the present invention utilizes a large level of transistor mismatch of approximately 10-20% as a desirable element of a most preferred design embodiment. The present invention's key advantage is its ability to convert mismatch error from a concerning and vexing problem into a desirable and useful means for achieving previously unachievable signal resolution and sensitivity, allowing designers of ordinary skill in the art to utilize smaller-sized components, and to relax physical layout constraints, which were engineering compromises that were required to be made in order to suppress mismatches in past design approaches. While the implication is that the RXF technique would work better in a deep submicron complementary metal-oxide semiconductor (CMOS) process with a large amount of mismatch, the random mismatch existing in the standard CMOS process may not be adequate. In situations where a “naturally” occurring mismatch is thought to be insufficient, it is now possible to increase the amount of error on purpose.

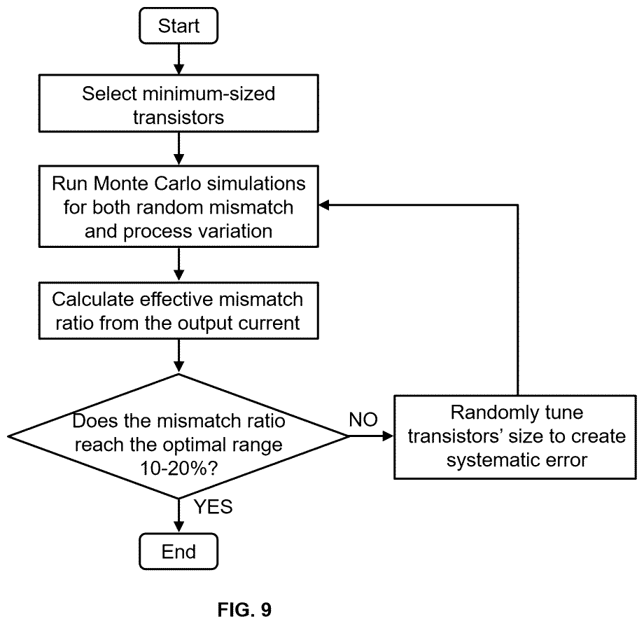

FIG. 9 illustrates a flow chart of a preferred procedure to tune a transistor mismatch ratio to achieve optimal super-resolution. Monte Carlo simulations can be used during the design process to evaluate the effective mismatch ratio. Random mismatch error (spread) is caused by variations and mismatches, while process (systematic) error (offset) is caused by non-ideal schematic and parasitic elements. If the random error in a design does not reach the most preferable level (i.e, 10-20%), then “artificial” mismatches may be created by arbitrarily tuning the individual unit transistors' size (W/L) by a range of from a few tens to a few hundred nanometers from the nominal value. The tuning values are randomly generated in a computer and manually added to each unit-transistor, (though this may influence the systematic error). The procedure is repeated with each bit of the IDAC and multiplier to the extent necessary. The procedure is ideally carried out with post-layout simulations, because the parasitic elements could result in creating additional systematic errors.

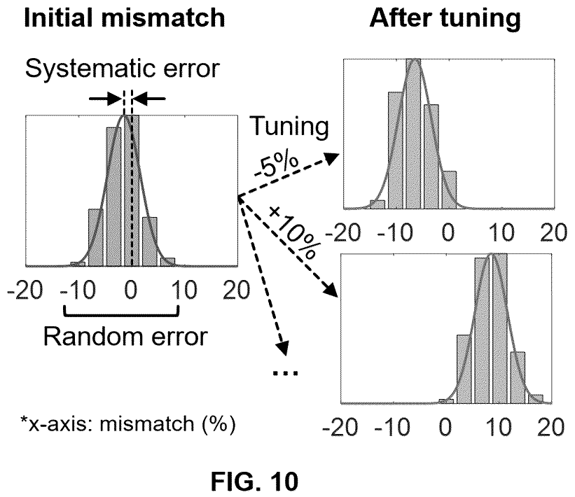

FIG. 10 illustrates the result of a Monte Carlo simulation, where the effective mismatch includes both the systematic error 1001 and random error 1002 components. The tuning process described above may introduce sought-after additional systematic errors, while not affecting the random error component.

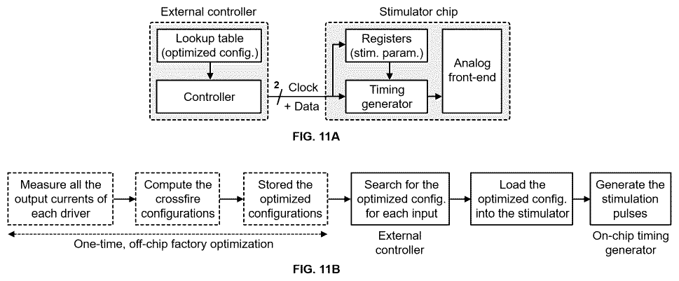

FIGS. 11A, and 11B show the present invention's stimulator control strategy, which consists of both an on-chip logic/memory process, and an off-chip logic/memory process. To achieve super-resolution, an RXF structure must be optimized. This is done with a one-time chip calibration procedure at the chip factory where all of the driver's output currents are measured. There are 2 (2⁵−1)·(2⁴−1)=930 non-zero values per driver to be measured, both cathodic and anodic. All the crossfire configurations are then computed. There are 2·(2⁵−1)·(2⁴·2⁴−1)=15,810 non-zero configurations per channel in a 2-way RXF structure. The optimized configuration associated with each desirable output current can be easily found by sorting through all the available values in a brute-force manner. While it is not an elegant solution, the optimization procedure only needs to be done once with the off-chip computation. The optimized configurations are then stored in an external lookup table. With a 10-bit effective resolution, the table size would be calculated to be 2·2¹⁰·(5+4+4)=26,624 bits (3.3 kB) per channel. An external controller maps each desirable output with the lookup table's optimized configuration during normal operation. The configuration is loaded into the stimulator chip via a transmission protocol consisting of a 10 MHz clock line and a data line.

The on-chip timing generator circuits produce stimulation pulses. This is essential to achieve a near-perfect synchronization of multiple drivers in a crossfire configuration. All of the stimulation parameters, such as pulse-width, IDAC, multiplier, polarity, and the like, are stored in integrated registers. The anodic and cathodic phases of a biphasic pulse can be independently configured to produce both symmetrical and asymmetrical stimulation with any ratio setting. New IDAC and multiplier configurations are loaded during the interphase delay. In a most preferred embodiment of the invention, a 16-bit register at a base clock of 10 MHz to control the pulse-width is used. This allows generating any timing from 0.01 msec to 6.56 msec with 0.1 μsec adjustment step. The adjustment step is also used to digitally compensate for the residual mismatch between the anodic and cathodic currents to ensure charge-balancing. This is achieved by tuning the anodic and cathodic pulse-width such that:

minimize|i_{A}·(t_{A}+Δt_{A})−i_{C}·(t_{C}+Δt_{C})|

The adjustment timings (Δt_{A}, Δt_{C}) are computed by an external controller based on the measured currents i_{A}, i_{C} and required pulse-width (t_{A}, t_{C}).

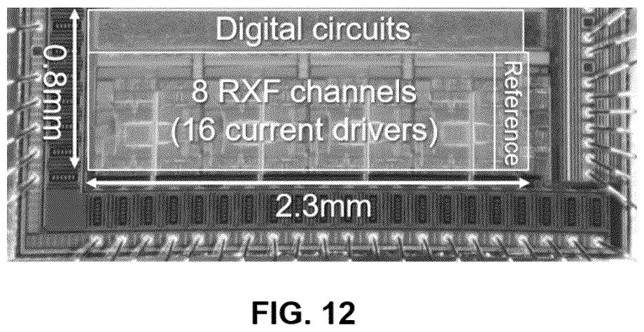

FIG. 12 shows a photomicrograph of a prototype chip of the most preferred embodiment of the invention, which was fabricated using the GlobalFoundries 0.18 μm BCDLite process. This embodiment utilizes isolated high-voltage LDMOS transistors, which are capable of supporting up to 30V. The disclosed chip contains 8 RXF channels (utilizing 16 current drivers) and occupies a core area of approximately 0.8 mm×2.3 mm. The chip's overall static power consumption is approximately 2.4 mW for the analog circuits at a 10V (±5V) supply and 1.6 mW for the digital circuits at a 1.8V supply. Most of the static power is generated from the high-voltage op-amps' bias current, which can be shut down during the interval that a channel is not in use.

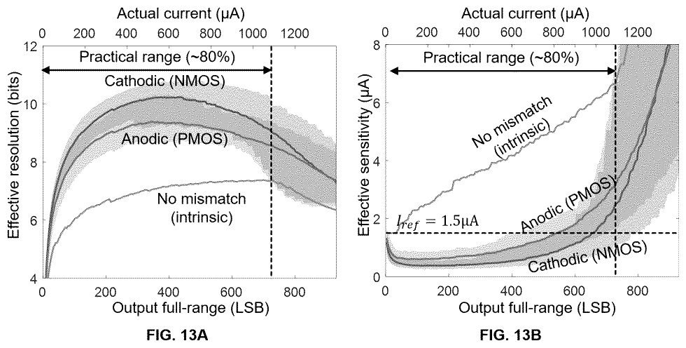

Chip measurement results. FIGS. 13A and 13B illustrate the measured results of the effective resolution and effective sensitivity for the above-described chip. The data were acquired from different channels and chips N_{Ch}=8, N_{IC}=10. The reference current was set to I_{ref}=1.5 μA, which translates to a practical output full-range of approximately 1.1 mA. The RXF technique of the present invention results in an effective super-resolution of 2-3 bits beyond the intrinsic baseline, conforming with the theoretical analysis. The effective sensitivity is well below the reference current across most of the output full-range, which was not possible to achieve in any prior art approach.

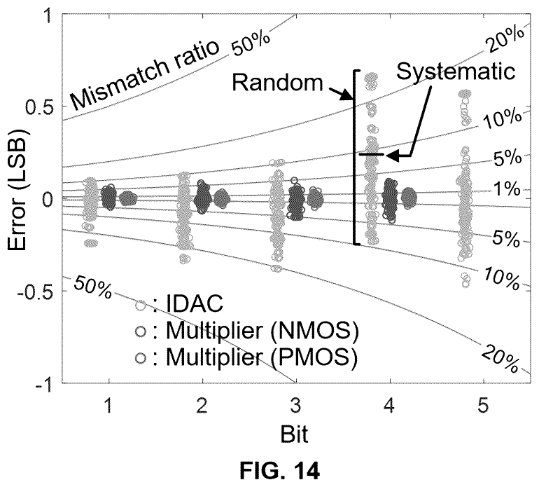

FIG. 14 illustrates the actual mismatch error computed from the measured current outputs for each bit of the IDAC and multiplier. The IDAC exhibits desirable random and systematic error, falling within the optimal 10-20% range.

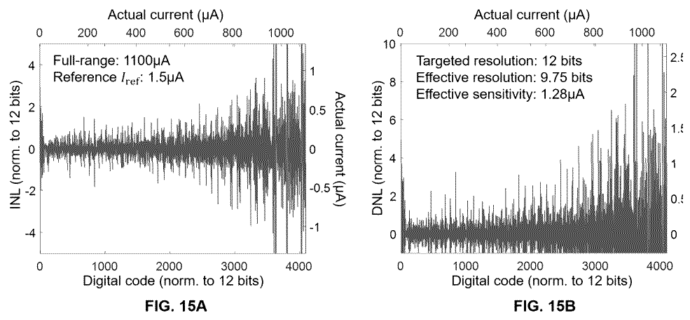

FIGS. 15A and 15B illustrate the measured integral nonlinearity (INL) and differential nonlinearity (DNL) of a stimulator channel. Here both the x-axis and y-axis are normalized to the targeted resolution of 12 bits over the 1.1 mA full range. The codes were optimized so that the outputs are always monotonic. The measured channel achieves an effective super-resolution of 9.75 bits and effective sensitivity of 1.28 μA. Unlike a conventional ADC/DAC, the INL and DNL of an RXF device are not symmetrical. Lower digital codes are more accurate because they contain more redundant configurations. The large spikes of INL/DNL data in higher digital codes (3500-4000 LSB) and the brief peak in the area of 0-50 LSB are associated with regions where there are not enough redundant configurations and their distribution is accordingly sparse.

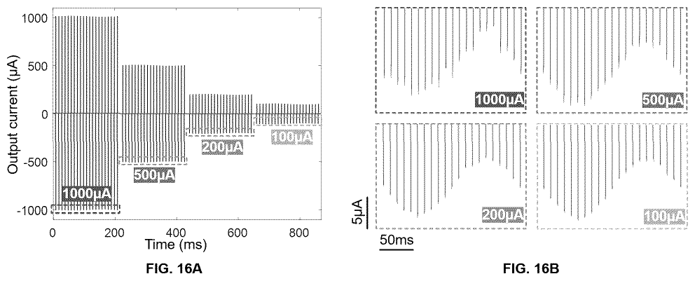

FIG. 16A illustrates examples of measured output current using, for example, a 1 kΩ resistive load. In this test, the chip generated a train of biphasic stimulation pulses at various output current levels of 100, 200, 500, and 1000 μA. Each pulse was accurately modulated to produce a sinusoidal waveform with a 5 μA ac amplitude. FIG. 16B zooms in to show greater detail of the pulse trains. The results indicate that the output at lower current levels is more accurate than the output at higher current levels, i.e., the sinusoidal waveforms of 100 or 200 μA are visibly less distorted than the sinusoidal wave forms of 500 or 1000 μA. Nevertheless, the waveform deviation at 1000 μA level is still within an accuracy of ±1 μA.

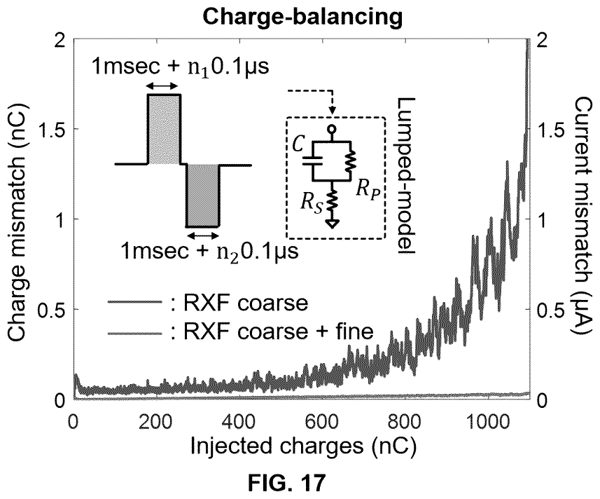

FIG. 17 illustrates a detail of the measured charge-balancing characteristics of a stimulator channel, previously alluded to in FIG. 1B. In this embodiment there were used a 1 msec pulse-width, anodic (positive) leading pulses, cathodic (negative) trailing pulses, and a lumped electrode model expressed as as C=0.5 μF, R_{S}=1 kΩ, R_{P}=10MΩ. There are two levels of charge-balancing, coarse and fine, both of which are digitally calculated by an external controller based on the measured currents and optimized configurations. The coarse calibration involves selecting the optimal IDAC and multiplier configuration of the second phase with a current amplitude matching the first phase. This process is done during the one-time factory optimization based on the measured current of every RXF configuration. Both the IDAC and multiplier values are adjusted during the inter-phase delay and require less than 10 μsec settling time. The fine calibration involves digitally tuning the second phase's pulse-width with 0.1 μsec steps to further compensate for any residual mismatch between the absolute amplitudes of the anodic and cathodic currents. With coarse calibration alone, the current mismatch is less than 0.2% across the entire output full-range. Again, lower current levels have more accurate matching. When combined with fine calibration, the overall charge mismatch is reduced to an insignificant degree ($<0.005%$).

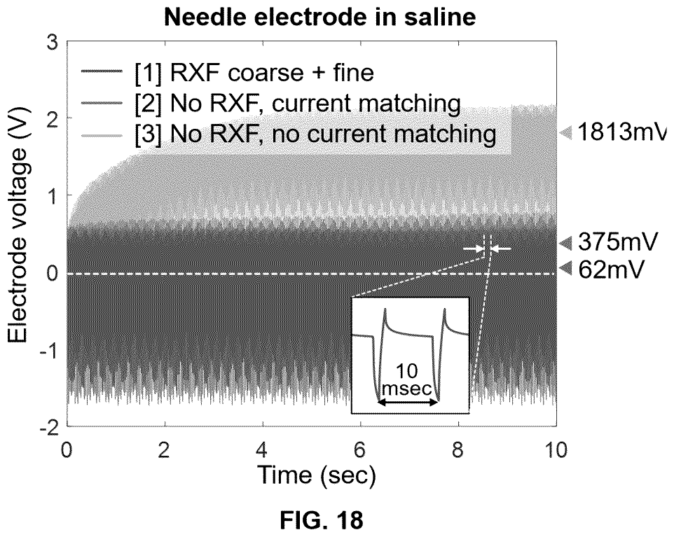

FIG. 18 illustrates the results of an aqueous environment experiment to verify the stimulator's charge-balancing characteristics in an aqueous saline solution. A pair of stainless-steel needle electrodes were submerged in a phosphate-buffered saline (PBS) preparation. The electrode's impedance was measured at 7.4 kΩ at 1 kHz. The stimulus is a train of cathodic leading, biphasic, symmetric pulses, with 1 msec pulse-width, and a targeted 200 μA current amplitude. The stimulation rate was 100 Hz, which approaches the upper bound of a most preferred embodiment application for the device. The 10 msec pulse-to-pulse spacing was selected since that rate does not allow sufficient time for the electrode to naturally discharge. In the first measurement, RXF with both coarse and fine calibration was used, and after 1000 pulses, the electrode's residual voltage stabilized at about 62 mV. The estimated current amplitudes were estimated and set at [−200.1, 200.0] μA, and 0.5 μsec was added to the second phase. In the second measurement, only one multiplier (but with no RXF) was used, to match the anodic and cathodic current with available configurations, and the residual voltage reached 375 mV and continued to increase. The estimated current amplitudes are [−201.2, 201.6] μA. In the third measurement, neither RXF nor current matching was used, and the residual voltage quickly reached 1813 mV, saturating the electrode interface as water began to undergo electrolysis. Current amplitudes were [−223.2, 230.7] μA.

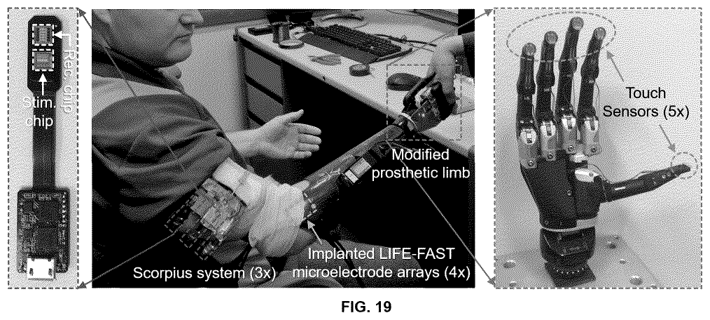

FIG. 19 photographically illustrates a neuroprosthesis experimental setup, which demonstrates the need for a high-resolution, fully-integrated neurostimulator, and this need is met by the chip architecture utilized in the present invention. The experiment is designed to restore somatosensation in a transradial amputee using electrical microstimulation while simultaneously acquiring nerve signals and generating new microstimulations that are re-calibrated in response to the processing of acquired nerve signals, in order to control a prosthetic hand's movements. A most preferred embodiment of a neurostimulator chip component is the essential part of the Scorpius neuromodulation system, a proprietary system known to those of ordinary skill in the art, that has both recording, and stimulation functions. The design and specifications of the Scorpius system are reported in Nguyen & Xu et al. A Bioelectric Neural Interface Towards Intuitive Prosthetic Control For Amputees. Journal of Neural Engineering, 17(6), 066001. (2020), the entire disclosure of which is incorporated herein by reference. Three Scorpius devices were used in the setup shown in FIG. 19, which can simultaneously address 24 independent stimulation channels. Four longitudinal intrafascicular electrode (LIFE) arrays were implanted into the patient using the microsurgical fascicular targeting (FAST) technique. The FAST-LIFE microelectrodes target discrete fascicles in the median and ulnar nerves. The design and characteristics of the electrodes are reported by Cynthia K. Overstreet et al Fascicle specific targeting for selective peripheral nerve stimulation. J. Neural Eng. 16 066040 (2019), the entire disclosure of which is incorporated herein by reference. The electrode's wires penetrated through the patients' skin and were connected to the Scorpius devices via standard Omnetics nano-connectors. The prosthesis was a heavily modified i-Limb Access hand (Touch Bionics, Livingston, UK). The prosthetic hand was equipped with touch sensors (Interlink Electronics, CA, USA) at the fingertips. In this most preferred embodiment of the present inventions, the hand's driver was replaced with a customized controller using the ESP32-WROOM-32 module (Espressif Systems, Shanghai, China). The ESP32 sampled force readouts from the sensors at 50 Hz and relayed data to the host server via Bluetooth. The host server used the sensor readouts to modulate the stimulation pulses' current amplitude to create various levels of touch sensation in the patient.

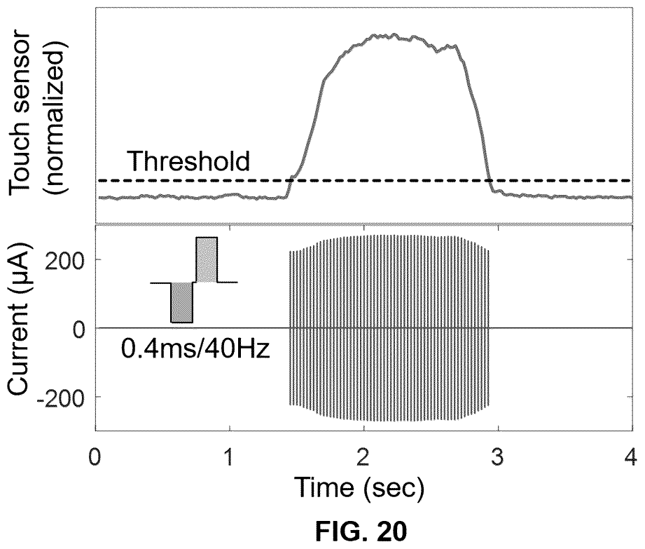

Turning now to FIG. 20, there is shown the resultant stimulation pattern as a function of elapsed time, as the prosthesis touched and released an object, which stimulation signal was applied to an electrode with clear sensory precepts acquired from previous mapping experiments. The stimulation pattern was a train of cathodic-leading, biphasic, symmetric pulses with a 0.4 msec pulse-width, at a 40 Hz rate. The current amplitude was modulated to be proportional to the applied force. The pulses were only generated when the sensor readout was above a certain threshold. The neurostimulator's super-resolution was essential for modulating the current amplitude with 1 μA accuracy at any given threshold within a 10-110 μA range. In one particular electrode embodiment shown in FIG. 20, the current amplitude ranged from 220 μA to 280 μA. The lower threshold 220 μA is the smallest current amplitude at which the patient can just barely begin to perceive sensation, while the higher threshold 280 μA is the highest current amplitude at which the patient can comfortably receive and tolerate signals without experiencing pain. Thus, it is essential to produce an accurate current amplitude within this range in order to deliver continuous and desirable sensory feedback from light to strong touch. It also noteworthy that the current thresholds vary widely across electrodes, even for those within the same microelectrode array. The thresholds may be as low as 15 μA and as high as 1000 μA. However, for any specific electrode, the working range (i.e., lower to higher threshold) is most preferably and 50-100 μA.

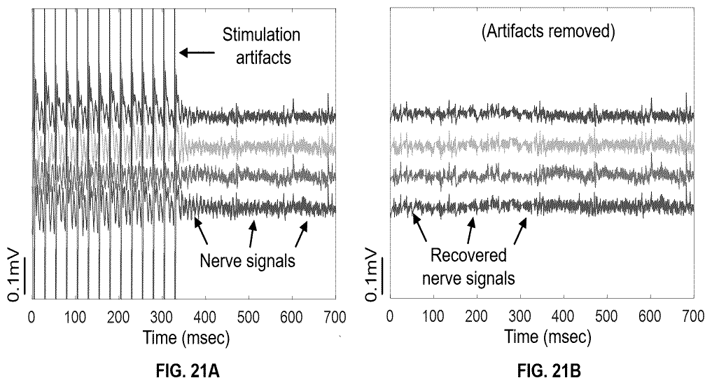

FIGS. 21A and 21B illustrates neural recordings acquired by the above described Scorpius system while the amputee patient flexed one of the prosthetic “phantom” fingers, and the resultant stimulation signal from the prosthetic finger's tip touchpad was delivered to an adjacent electrode. A charge-balanced neurostimulator also played an important role in motor decoding experiments with simultaneous somatosensory feedback. The data show that the stimulation artifacts overlapped with the nerve signals. Charge-balancing helps reduce the impact of artifacts and prevents long-term charge accumulation, which could hinder the recorder's operation. The results show that the artifacts may be removed to recover most of the nerve data for decoding the amputee's motor intentions. The artifacts are removed offline using the template matching method for demonstration purposes. A brief duration of 2-3 msec at the onset of each stimulation pulse is removed and replaced with a straight line because the recorder's input is fully saturated.

Super Resolution Digital-to-Analog Converters Based on Redundant Sensing.

Systems and methods for producing digital-to-analog converters (DACs) that provide super-resolution without post-processing and in the presence of mismatch error are now provided by the present disclosure.

The UN grouping-based SR technique detailed here is fundamentally different from certain previous approaches because it does not involve reconstructing the missing information nor rely on any statistical properties of the input data. The SR capability has been embedded in the sensor's endogenous structure once fabricated thanks to its redundant architecture. This “hidden” potential must be revealed by optimization in order to achieve SR data acquisition. The optimization process only needs to be done once for each sensor and is independent of the input signals. Once optimized, the sensor can capture any type of signals at super-resolved resolution regardless of their statistical distributions. For compressive sensing or other data-driven methods including most existing machine learning based techniques, optimization or approximation is performed during the reconstruction process, after the low-resolution data have been acquired. In contrast, for the UN grouping method, optimization is performed on the sensor before acquiring any data and the fine-detailed information content of the input signal is never lost during quantization. This is achieved not only because of the RS architecture itself but also by elegantly manipulating mismatch error—an undesirable precision-limiting factor in conventional designs.

In the following “Super Resolution” section, the mechanisms of the new theory to facilitate SR in a RS architecture are presented. The Monte Carlo method is used to demonstrate the advantages of the UN technique. The Monte Carlo analysis is an effective and widely-used methodology when traditional proofs are too complex or not feasible, especially in this case which can be shown to be a NP-hard optimization problem. The analysis is performed at both abstract-level where a simple probabilistic distribution of the components is assumed and circuit-level where all the non-ideal factors due to process variation are considered. A component is an assembly of one or more unit cells that behaves like a single entity. The component set (which may also be referred to as a set of components) is the collection of components used by the sensor to generate its internal reference. For example, a binary-weighted sensor has the component set of {1, 2, 4, . . . , 2^{N-1}} that is assembled from 2^{N}−1 identical unit cells where a unit cell has the weight of 1 (unit). The unit cells can output an electrical signal such as a voltage or a current. The results demonstrate an extra 8-9 bits resolution or 256-512× precision can be accomplished on top of a 10-bit quantizer at 95% sample space. In the “Practical Consideration And Applications” section, potential applications and practical considerations of the proposed SR technique in fully-integrated miniaturized biomedical devices where the structure's complexity can be mitigated by approximation or conveniently circumvented are described. An example design is shown where the UN grouping technique can be applied to boost the resolution of the current DAC in a neurostimulator, giving more precise control of the output stimulation current.

Super-Resolution; Quantization and Mismatch Error

Quantization is a process of mapping a continuous set (analog) to a finite set of discrete values (digital). Without loss of generality, it can be assumed that a No quantizer divides the continuous interval [0, 1) into 2^{N 0} partitions defined by a set of references θ₀≤θ₁< . . . ≤θ_{2 N 0} where each partition is mapped onto a digital code d ranging from 0 ro 2^{N 0}−1. It can be shown that H_{N 0}<N₀ for all values of reference θ_{d}. Equality occurs only when 2^{N 0} references are equally spaced, i.e., ∀i,j:θᵢ₊₁−θᵢ=θⱼ₊₁−θⱼ. This fundamental maximum value of entropy is referred as the Shannon limit, where the device's effective resolution is theoretically bounded only by its intrinsic quantization error.

In practice, the quantizer's precision is also affected by the randomly occurred mismatch error, resulting in the undesirable deviation of the references and degradation of entropy. For example, some integrated ADC or DAC chips generate their references by arrays of identical elementary components regarded simply as unit cells. A N₀-bit device generally has 2^{N 0}−1 unit cells which could be miniature capacitors, resistors or transistors. The random mismatch of individual unit cells due to variations of the fabrication process and other non-ideal factors is one of the primary sources of mismatch error that could significantly deteriorate the device's precision.

To effectively control the unit cells, the cells are generally grouped into bundles regarded simply as components. Grouping significantly reduces the number of control signals required. For example, with the conventional binary-weighted method, 2^{N 0}−1 unit cells are arranged into No components with the nominal weight of {2⁰, 2¹, . . . , 2^{N 0-1}}. Such system is orthogonal because with No binary control signals, i.e., 0/1 bits, 2^{N 0} references corresponding to each digital code in [0, 2^{N 0}−1] can be uniquely created by selecting and assembling the components according to the binary numeral system.

Redundant Sensing. Redundant Sensing (RS) is a design framework that aims at engineering redundancy for enhancing the system's performance regarding accuracy and precision, instead of reliability and fault-tolerance like other designs. A practical RS implementation must satisfy two criteria, namely representational redundancy (RPR) and entangled redundancy (ETR). RPR refers to a non-orthogonal scheme of information representation where every outcome in the sample space is encoded by numerous distinct system configurations. Each configuration responses differently to mismatch error such that in any given instance, there almost always exists one or more configurations that have smaller errors than the conventional representation.

ETR refers to the implementation of the RS structure such that the statistical distribution of different system configurations is partially correlated (i.e., entangled) allowing a large degree of redundancy without incurring excessive resource overhead. ETR should be differentiated from conventional replication-based method to realize redundancy where the degree of redundancy is linearly proportional to the resource utilization. While using the same amount of physical resource (i.e., 7 unit cells), in the RS structure, each digital code can be created by multiple distinct assemblies of components, each expresses a different, partially correlated distribution with respect to random mismatch error. This redundant system of information representation has been shown to suppress mismatch error by allowing searching for the optimal component assembly with the least error with respect to each and every digital code. The redundant mechanism can be elegantly exploited to realize an effective resolution beyond the conventional limit of No bounded by quantization error.

Code Diffusion. Mismatch ratio am is defined as the standard deviation of each unit cell which is assumed to have a Gaussian distribution with unity mean. In the absence of mismatch error or σₘ=0, regardless how the unit cells are grouped and assembled, an array of 2^{N 0}−1 identical units can only generate a finite number of references. Code diffusion is the property of an RS structure where the actual value of its internal references spreads into the neighbor sample space because of random mismatch error. With sufficient level of mismatch ratio, the reference's probability density function covers almost all the sample space with relatively even chances. It is also interesting to point out that mismatch error, which is conventionally regarded as an undesirable non-ideal factor, is the crucial element that enables SR. Maximal effectiveness of SR is obtained only when the mismatch ratio reaches a certain level (e.g., ^{˜}10%) which would be considered excessively large in many ordinary applications. Such mechanism is only possible because the number of distinct references that can be generated by a RS structure is significantly larger than the ingrinsic cardinality due to redundancy. Furthermore, not only the number of different component assemblies but also the mutual correlation between them play an important role. Ideally, the assemblies would be spread evenly across all the sample space to have the maximum chance of approximating Θ_{N k}. This characteristic is determined by the device's internal architecture, i.e., how the components are designed.

Grouping Method. The grouping method is a preferred way, where unit cells are arranged into components. Almost all conventional designs can be categorized as binary-weighted (BW) structures where the quantization partitions are uniquely encoded according to the binary numeral system. In contrast, the proposed RS architecture employs a different strategy to realize redundancy with both RPR and ETR properties. There is no limitation to how the unit cells are grouped. While the grouping method does not alter the number of unit cells, thus has little effect on the resource constraints, it determines the system's endogenous architecture and greatly affects the references' number and distribution. The design of grouping method differentiates one redundant structure from another.

Beyond the Shannon Limit. SR in the context of this disclosure should be understood as a resource-constraint problem. The precision of a sensor consists of 2^{N 0}−1 unit cells was previously thought to be bound by the Shannon limit of No determined by quantization error. By arranging the unit cells in a specific manner to realize a redundant structure and exploiting the statistical property of random mismatch error, an effective resolution beyond this conventional “limit” is achieved.

As the analysis of code diffusion suggested, the best performance of SR is obtained with the mismatch ratio being above ^{˜}10%. Both HS and UN grouping method offers 3-4 bits increase of effective resolution or 8 x-16× enhancement of precision. The entropy's STD is less than 0.2-bit within 10-50% mismatch ratio where the UN method has a marginally better outcome. These results suggest that the solution for SR is consistent which in practical applications, will translate to the good yield of the device under random error.

Furthermore, the consistency of the mechanism implies that mismatch error may not need to be truly “random”. In certain application, 10% random deviation may seem unrealistic. Instead, the deviation can be intentionally added to the structure during the design process. Even if these artificial pseudo-random deviations could carry a certain level of error, the consistency of SR mechanism guarantees that a solution can always be found. Furthermore, the proposed SR method can also be applied to enhance the performance of numerous biomedical devices that employ a DAC. For example, electrical neurostimulators generally require a DAC to generate an internal reference current. A higher resolution DAC is always desirable as it gives more precise control of the stimulation current in a wider range, which could imply better modulation of different neural circuits. In another example, many ultrasound imaging modalities employ a DAC in their transmission stage to generate the necessary analog signals. High-precision commercial DACs up to 12 bits and beyond have been used in various systems to facilitate their operation. Implementing such high-precision DACs (10-12 bits) on-chip is generally challenging and expensive because they occupy large silicon area, especially in high-voltage processes (>30 V). The proposed UN method could greatly benefit these designs by help achieving a similar resolution with a much lower cost. The results that the proposed SR mechanism can be utilized to greatly enhance the performance of a high-precision device by exploiting the natural mismatch of the transistors.

Moreover, unlike the ADC example, the neurostimulator's operations are always governed by an external controller during normal operation. The controller regularly communicates with the neurostimulator to update its parameters and trigger its function when needed. Subsequently, the optimal system setting at every DAC output can be simply determined upfront via foreground calibration and saved on an external memory, i.e., a look-up table, which is accessed by the controller at any instant. This effectively circumvents the computational-hard problem by diverting it into a memory-hard problem which could be more easily handled in certain circumstances. For instance, assuming a targeted SR of 16-bit is to be achieved with 20 components, storing all the optimal configurations would require 2¹⁶×20=1.3·10⁶ bits or 163 KB of memory per DAC—a trivial amount for an off-chip flash memory. A DAC converter device can include a number of DAC channels. In some embodiments, the DAC device can include sixteen channels. One or more controllers can be coupled to the channels included in the DAC device in order to control the channels as described above. In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media. It should be noted that, as used herein, the term mechanism can encompass hardware, software, firmware, or any suitable combination thereof.

This disclosure presents a new interpretation of the RS architecture that allows quantization or de-quantization processes to achieve an effective resolution many folds beyond the limitation that their resource constraints commonly permit. Using Monte Carlo simulations, it is shown that SR is feasible by elegantly exploiting the statistical property called “code diffusion” that is unique to a redundant structure in the presence of random mismatch error. By applying the UN method on a 10-bit device, a profound theoretical increase of 8-9 bits effective resolution or 256-512× enhancement of precision at 95% sample space is demonstrated. The UN grouping method can be applied to various fields of biomedical imaging and data acquisition instrumentation, especially low-power fully-integrated sensors and devices where higher resolution is always desired, as well as other applications such as audio and video processing, data communications including wire/wireless data transmission and/or data storage, remote sensing such as radar, sonar, ultrasound and/or infrared sensing, sensors and actuators used in robotics, etc.

Thus, the present disclosure provides systems and methods for producing digital-to-analog converters that provide super-resolution without post-processing and in the presence of mismatch error.

The present invention has been described in terms of one or more preferred embodiments, and it should be appreciated that many equivalents, alternatives, variations, and modifications, aside from those expressly stated, are possible and within the scope of the invention.

While the above description contains much specificity, these should not be construed as limitations on the scope of any embodiment, but as exemplifications of the presented embodiments thereof. Many other alternative embodiments and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention will not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are, unless otherwise stated, used in a generic and descriptive sense only and not for purposes of limitation, the scope of the invention therefore not being so limited. Moreover, the use of the terms first, second, etc. do not denote any order or hierarchy of importance, but rather the terms first, second, etc. are used to distinguish one element from another. Furthermore, the use of the terms a, an, etc. do not denote a limitation of quantity, but rather denote the presence of at least one of the referenced items.

While the invention has been described, exemplified, and illustrated in reference to certain preferred embodiments thereof, those skilled in the art will appreciate that various changes, modifications, and substitutions can be made therein without departing from the spirit and scope of the invention. It is intended, therefore that the invention be limited only by the scope of the claims which follow, and that such claims be interpreted as broadly as is reasonable.

What is claimed is:

1. A neurostimulator system, comprising:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a combined, crossfiring, output of said current drivers that produces a redundant sensing structure that produces accurate current pulses with an effective super-resolution accuracy beyond ordinary limitations imposed by physical constraints of materials in said system.

2. The system as claimed in claim 1, wherein said redundant structure is configured so as to achieve a super-resolution signal accuracy outcome by applying the effects of random mismatch error function to said system, with the proviso that mismatch avoidance and mismatch compensation functions are not applied in achieving said super-resolution signal accuracy outcome.

3. The system as claimed in claim 2, wherein said random mismatch error function is configured so as to select and tune transistor size to achieve a desired mismatch ratio of 10% to 20%.

4. The system as claimed in claim 3, additionally comprising an on-chip timing generator.

5. The system as claimed in claim 4, additionally comprising both on-chip and off-chip components configured so as to retrievably store calculated optimal transistor configurations obtained through foreground calibration, and which configurations can be retrieved and read by said on-chip timing generator so as to produce signal output with super resolution accuracy.

6. The system as claimed in claim 5, wherein said on-chip component is a memory chip component of said system.

7. The system as claimed in claim 5, where said off-chip component is a look-up table component of said system.

8. The system as claimed in claim 1, additionally comprising an external controller configured so as to ensure charge-balancing that is achieved by digital compensation for residual mismatch between said anodic and cathodic currents.

9. The system as claimed in claim 8, wherein said charge balancing is further characterized as being coarse level charge balancing.

10. The system as claimed in claim 8, where said charge balancing is further characterized as being fine level charge balancing.

11. The system as claimed in claim 1, configured so as to modulate the neurostimulation intensity of said crossfiring redundant signal output to create various levels of somatosensorial signal outputs of from light to strong touch in real time in a neuroprosthesis device.

12. The system as claimed in claim 2, wherein said random mismatch error function is configured so as to select and tune diode size to achieve a desired mismatch ratio.

13. The system as claimed in claim 2, wherein said random mismatch error function is configured so as to select and tune resistor size to achieve a desired mismatch ratio.

14. The system as claimed in claim 2, wherein said random mismatch error function is configured so as to select and tune capacitor size to achieve a desired mismatch ratio.

15. The system as claimed in claim 2, whereby application of the effects of random mismatch error function in said system is configured so as to be applied to extremely large mismatches to achieve super-resolution over 10-fold beyond intrinsic resolution of said design imposed by physical constraints of materials in said system.

16. A high-resolution constant-current stimulator neuroprosthesis neurostimulator chip, comprising:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a crossfiring, combined output of said current drivers that produces a redundant structure to produce accurate current pulses with an effective super-resolution beyond limitations of physical constraints of mismatch error in materials in said system.

17. An electrical neuromodulation neurostimulator chip for the generation of neurostimulation signals in a neuroprosthesis, wherein said chip comprises:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a crossfiring, combined output of said current drivers that produces a redundant structure to produce accurate current pulses with an effective super-resolution beyond limitations of physical constraints of materials in said system.

18. A method of rehabilitating an amputee by fitting said amputee with a tactile-sensitive neuroprosthesis comprising the neurostimulator chip of claim 17.

19. The method of claim 18, wherein said neuroprosthesis is a prosthetic forearm and hand.

20. The method of claim 18, wherein said neuroprosthesis is a prosthetic hand.

21. A digital-to-analog converter device comprising:
a set of components, each component included in the set of components comprising a number of unit cells and at least one component including a number of unit cells that is not a power of two;
a plurality of switches, each switch included in the plurality of switches being coupled to a component included in the set of components;
an output electrode coupled to the plurality of switches, the digital-to-analog converter device being configured to output an output signal at the output electrode; and
a controller coupled to the plurality of switches and configured to: receive a desired output current;
determine an anodic component configuration comprising at least one component included in the set of components based on the desired output current;
determine a cathodic component configuration comprising at least one component included in the set of components based on the desired output current; and
cause a current pulse to be output at the output electrode based on the anodic component configuration and the cathodic component configuration.

22. The digital-to-analog converter device of claim 21, wherein the current pulse includes a positive current pulse and a negative current pulse.

23. The digital-to-analog converter device of claim 21, wherein the controller comprises a memory comprising a set of positive current values and negative current values associated with a set of component configurations, the anodic component configuration and the cathodic component configuration being included in the set of component configurations.

24. The digital-to-analog converter device of claim 21, wherein the anodic component configuration includes at least one component not included in the cathodic component configuration

25. The digital-to-analog converter device of claim 21, wherein an effective resolution of the digital-to-analog converter device is at least four times greater than an intrinsic resolution of the digital-to-analog converter device.

26. The digital-to-analog converter device of claim 25, wherein the effective resolution of the digital-to-analog converter device is equal to a Shannon entropy of the digital-to-analog converter device, and the intrinsic resolution is equal to log base two of the number of unit cells plus one.

27. The digital-to-analog converter device of claim 21, wherein the digital-to-analog converter device is included in a neurostimulator device.

28. The digital-to-analog converter device of claim 21, wherein a first unit cell size associated with a first unit cell included in the set of components is different than a second unit cell size associated with a second unit cell included in the set of components, the first unit cell size comprising a length and width of the first unit cell.

29. The digital-to-analog converter device of claim 28, wherein the first unit cell size and the second unit cell size are associated with a transistor process size.

30. The digital-to-analog converter device of claim 21, wherein each unit cell comprises at least one transistor.

31. The digital-to-analog converter device of claim 21, wherein an effective resolution of the digital-to-analog converter device is at least two hundred times greater than an intrinsic resolution of the digital-to-analog converter device for at least ninety-five percent of a sample space of the digital-to-analog converter device.

32. The device of claim 1, additionally comprising a digital-to-analog converter device comprising:
a set of components, each component included in the set of components comprising a number of unit cells, each unit cell being associated with a unit cell size indicating manufacturing specifications of the unit cell;
a plurality of switches, each switch included in the plurality of switches being coupled to a component included in the set of components; and
an output electrode coupled to the plurality of switches, the digital-to-analog converter device being configured to output an output signal at the output electrode, wherein a first unit cell size associated with a first unit cell included in the set of components is different than a second unit cell size associated with a second unit cell included in the set of components.

33. The digital-to-analog converter device of claim 32, wherein the unit cell size comprises a length value and a width value.

34. The digital-to-analog converter device of claim 32, wherein the unit cell size is associated with a transistor process size.

35. The digital-to-analog converter device of claim 32, wherein at least one component included in the set of components includes a number of unit cells that is not a power of two.

36. The digital-to-analog converter device of claim 32, wherein the digital-to-analog converter device is a current digital-to-analog converter.

37. The digital-to-analog converter device of claim 36, wherein the digital-to-analog converter device further comprises a controller coupled to the plurality of switches, the controller configured to receive a desired output current;
determine a component configuration based on the desired output current and a predetermined output current value measured at the output electrode, the predetermined output current value associated with the component configuration; and
cause a current pulse to be output from the digital-to-analog converter device based on the component configuration.

38. A method for determining manufacturing parameters for a digital-to-converter device in a neurostimulator system, comprising:
the use of at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a combined, crossfiring, output of said current drivers that produces a redundant sensing structure that produces accurate current pulses with an effective super-resolution accuracy beyond ordinary limitations imposed by physical constraints of materials in said system comprising a set of components, each component included in the set of components including at least one unit cell, and each unit cell being associated with a unit cell size, the steps of the method comprising:
determining a required mismatch error value for the unit cells included in the component set based on a targeted effective resolution value;
determining an initial unit cell size based on the required mismatch error value;
setting the unit cell size of each unit cell included in the component set to be equal to the initial unit cell size;
determining an effective resolution of the digital-to-analog converter device by performing simulations;
determining that the effective resolution is below the targeted effective resolution;
adjusting the unit cell size of one or more unit cells included in the component set in response to determining that the effective resolution is below the targeted effective resolution; and
providing each unit cell size associated with each unit cell to a manufacturing facility.

39. The method of claim 38, wherein the targeted effective resolution is at least four times higher than an intrinsic resolution of the digital-to-analog converter device.

40. The method of claim 38, wherein the unit cell size comprises a length value and a width value, and wherein each unit cell comprises at least one transistor.

41. The method of claim 39, wherein at least one component included in the set of components includes a number of unit cells that is not a power of two.
